SEQUENCE LISTING (33 sequences)

SEQ ID NO: 1 - protein, 199 aa, Bos taurus
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 2 - protein, 199 aa, synthetic construct
RPKHPIKHQGLPQEVLNENLLRFFVAPFPEVFGKEKVNELSKDIGDEDTEDQAMEDIKQMEAEDIDDDEEIVPNDVEQKHIQKEDVPSERYLGYLEQLLRLKKYKVPQLEIVPNDAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 3 - protein, 209 aa, Bos taurus
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 4 - protein, 209 aa, synthetic construct
RELEELNVPGEIVEDLDDDEESITRINKKIEKFQDEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 5 - protein, 386 aa, Bacillus subtilis
MLNWTPKLQSLSTPFAKLSTAAITPTGPAIREATLQFEKYAQEGACWQMMNDALTSLACSLKPGTTIKGKWNGNTYTLRKQLGKGANGIVYLAETSDGHVALKVSDDSLSITSEVNVLKSFSKAQSVTMGPSFFDTDDAYIPSANTKVSFYAMEYIKGPLLLKYVSDKGAEWIPVLMIQLLSSLSVLHQQGWIFGDLKPDNLIVTGPPARIRCIDVGGTTKEGRAIKEYTEFYDRGYWGYGTRKAEPSYDLFAVAMIMINSVHKKEFKKTNQPKEQLRSLIEGNPLLQKYKKALFSALNGDYQSADEMKKDMLDAGQKAAQRKQPIKASPQPATRQRQQKPRQGKITKTRYTPKQKPAKSGGLFETTLIVISVLALYFAYIIFFLI

SEQ ID NO: 6 - protein, 321 aa, Bacillus subtilis
MMNDALTSLACSLKPGTTIKGKWNGNTYTLRKQLGKGANGIVYLAETSDGHVALKVSDDSLSITSEVNVLKSFSKAQSVTMGPSFFDTDDAYIPSANTKVSFYAMEYIKGPLLLKYVSDKGAEWIPVLMIQLLSSLSVLHQQGWIFGDLKPDNLIVTGPPARIRCIDVGGTTKEGRAIKEYTEFYDRGYWGYGTRKAEPSYDLFAVAMIMINSVHKKEFKKTNQPKEQLRSLIEGNPLLQKYKKALFSALNGDYQSADEMKKDMLDAGQKAAQRKQPIKASPQPATRQRQQKPRQGKITKTRYTPKQKPAKSGGLFETTLI

SEQ ID NO: 7 - protein, 256 aa, Bacillus subtilis
MALKLLKKLLFDRPLKNGVILNHQYKIEECLGMGGYGLVYLCTDILAQTPYVLKQLRPTKAKKEKEKVRFQQEIKLLKNIHHPQIPGFIDEFIIDGQAYYVMQFIEGENIEELLFFRKQPFTELMALQLISQLLEIIEYLHDRLIFHSDIRTPNIIINDGRLCLIDFGLAKQLTPEEMEEIKVRKQDDFFDLGETLLFLLYSQYKGKKKKNGTWLEELTLTKEVTLLLKRLLGIEEEYQHTASIREDLNRAIQSVT

SEQ ID NO: 8 - protein, 390 aa, Bacillus subtilis
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQ

SEQ ID NO: 9 - protein, 283 aa, Corynebacterium glutamicum
MYEVLGVIAHGGMGWIYLANDRNVSGRIVVLKGMMAQSSVQDQGTAEAEREFLADITHPGIVKAYNFIDDPRVPGGFIVMEYVNGPSLKDRCKAQPDGVLRVDLAIGYILELLPAMDYLHQRGVVYNDLKPENVIATEDQVKLIDLGAVTGIGAFGYIYGTKGFQAPEVATHGPSISSDIFTIGRTLAALTMPLPVEDGVLAPGIPSPKNSPLLRRHLSFYRLLQRATADDPQHRFRNVSELRTQLYGVLREILAVRDGKQYPPQHSLFSPQRSTFGTKHLVF

SEQ ID NO: 10 - protein, 185 aa, Homo sapiens
MRLLILTCLVAVALARPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 11 - protein, 230 aa, Camelus dromedarius
MKLLILTCLVAVALARPKYPLRYPEVFQNEPDSIEEVLNKRKILELAVVSPIQFRQENIDELKDTRNEPTEDHIMEDTERKESGSSSSEEVVSSTTEQKDILKEDMPSQRYLEELHRLNKYKLLQLEAIRDQKLIPRVKLSSHPYLEQLYRINEDNHPQLGEPVKVVTQEQAYFHLEPFPQFFQLGASPYVAWYYPPQVMQYIAHPSSYDTPEGIASEDGGKTDVMPQWW

SEQ ID NO: 12 - protein, 214 aa, Ovis aries
MKLLILTCLVAVALARPKHPIKHQGLSSEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 13 - protein, 214 aa, Capra hircus
MKLLILTCLVAVALARPKHPINHRGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 14 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 15 - protein, 214 aa, Bubalus bubalis
MKLLILTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSGKTTMPLW

SEQ ID NO: 16 - protein, 27 aa, Homo sapiens
MKFFIFTCLLAVALAHHEIKHSSSSSE

SEQ ID NO: 17 - protein, 193 aa, Camelus dromedarius
MKFFIFTCLLAVVLAKHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFTKKTELTEEEKDHQKFLNKIYQYYQTFLWPEYLKTVYQYQKTMTPWNHIKRYF

SEQ ID NO: 18 - protein, 223 aa, Ovis aries
MKFFIFTCLLAVALAKHKMEHVSSSEEPINISQEIYKQEKNMAIHPRKEKLCTTSCEEVVRNADEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 19 - protein, 223 aa, Capra hircus
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 20 - protein, 222 aa, Bos taurus
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 21 - protein, 222 aa, Bubalus bubalis
MKFFIFTCLLAVALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEDKNRLNFLKKISQHYQKFAWPQYLKTVYQYQKAMKPWTQPKTNVIPYVRYL

SEQ ID NO: 22 - protein, 226 aa, Homo sapiens
MKVLILACLVALALARETIESLSSSEESITEYKQKVEKVKHEDQQQGEDEHQDKIYPSFQPQPLIYPFVEPIPYGFLPQNILPLAQPAVVLPVPQPEIMEVPKAKDTVYTKGRVMPVLKSPTIPFFDPQIPKLTDLENLHLPLPLLQPLMQQVPQPIPQTLALPPQPLWSVPQPKVLPIPQQVVPYPQRAVPVQALLLNQELLLNPTHQIYPVTQPLAPVHNPISV

SEQ ID NO: 23 - protein, 232 aa, Camelus dromedarius
MKVLILACLVALALAREKEEFKTAGEALESISSSEESITHINKQKIEKFKIEEQQQTEDEQQDKIYTFPQPQSLVYSHTEPIPYPILPQNFLPPLQPAVMVPFLQPKVMDVPKTKETIIPKRKEMPLLQSPVVPFTESQSLTLTDLENLHLPLPLLQSLMYQIPQPVPQTPMIPPQSLLSLSQFKVLPVPQQMVPYPQRAMPVQAVLPFQEPVPDPVRGLHPVPQPLVPVIA

SEQ ID NO: 24 - protein, 222 aa, Ovis aries
MKVLILACLVALALAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLPPTVMFPPQSVLSLSQPKVLPVPQKAVPQRDMPIQAFLLYQEPVLGPVRGPFPILV

SEQ ID NO: 25 - protein, 222 aa, Capra hircus
MKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGPFPILV

SEQ ID NO: 26 - protein, 224 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 27 - protein, 224 aa, Bubalus bubalis
MKVLILACLVALALARELEELNVPGEIVESLSSSEESITHINKKIEKFQSEEQQQMEDELQDKIHPFAQTQSLVYPFPGPIPKSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 28 - protein, 182 aa, Homo sapiens
MKSFLLVVNALALTLPFLAVEVQNQKQPACHENDERPFYQKTAPYVPMYYVPNSYPYYGTNLYQRRPAIAINNPYVPRTYYANPAVVRPHAQIPQRQYLPNSHPPTVVRRPNLHPSFIAIPPKKIQDKIIIPTINTIATVEPTPAPATEPTVDSVVTPEAFSESIITSTPETTTVAVTPPTA

SEQ ID NO: 29 - protein, 182 aa, Camelus dromedarius
MKSFFLVVTILALTLPFLGAEVQNQEQPTCFEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 30 - protein, 192 aa, Ovis aries
MMKSFFLVVTILALTLPFLGAQEQNQEQRICCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 31 - protein, 192 aa, Capra hircus
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 32 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 33 - protein, 132 aa, Bubalus bubalis
YGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSDVIESVPETNTAQVTSTVV

(19) United States
(12) Patent Application Publication
(10) Pub. No.: US 2025/0206792 A1
(43) Pub. Date: Jun. 26, 2025

(54) METHOD TO PRODUCE PHOSPHORYLATED MILK PROTEINS IN MICROBES

(71) Applicant: Danmarks Tekniske Universitet, Kongens Lyngby (DK)

(72) Inventors: Suvasini Balasubramanian, Kongens Lyngby (DK); Peter Ruhdal Jensen, Kongens Lyngby (DK); Carsten Jers, Kongens Lyngby (DK); Golnaz Mobasseri, Göteborg (SE); Ivan Mijakovic, Göteborg (SE); Lei Shi, Göteborg (SE)

(21) Appl. No.: 18/390,266

(22) Filed: Dec. 20, 2023

(51) Int. Cl. C07K 14/47; C12N 1/20; C12N 9/12; C12R 1/19

(52) U.S. Cl. CPC C07K 14/4732; C12N 1/205; C12N 9/12; C12R 2001/19; C12Y 207/11001

(57) ABSTRACT

The present disclosure relates to methods for production of phosphorylated casein, phosphorylated phosphomimetic casein and phosphomimetic casein, as well as the use of such caseins.

### TECHNICAL FIELD

The present disclosure relates to methods for production of phosphorylated casein, phosphorylated phosphomimetic casein and phosphomimetic casein, as well as the use of such caseins.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created Dec. 20, 2023, is named “P6930US00-Sequence listing xml (40991059.1).xml” and is 36,041 bytes in size.

### BACKGROUND

Milk proteins, such as caseins and whey proteins are commonly found in mammalian milk. Caseins are some of the most valued protein sources, for their superior nutritional and functional attributes, and has a wide variety of uses, from being a major component of cheese, to the use of casein as a food additive for emulsification, texture and stabilization.

Methods for production of recombinant caseins have been known for many years (Jimenez-Flores et al, 1990, Thurmond et al, 1997), however these methods generally suffer from limitations such as poor yield ranges and expression of milk protein without the valued functional attributes. To this date, no method allowing for industrial production of recombinant caseins, which can overcome the expression limitations has been developed.

### SUMMARY

The invention is as defined in the claims.

In a first aspect the present disclosure concerns a system for producing one or more phosphorylated caseins, said system comprising:
i. a microbial host cell expressing:
   a. at least one recombinant casein selected from the group consisting of alpha-casein S1 (αS1), alpha-casein S2 (αS2), beta-casein and kappa-casein, and
   b. at least one microbial kinase;
      or
ii. a microbial host cell expressing at least one casein selected from alpha-casein (αS1, αS2), beta-casein or kappa-casein, and
   a composition comprising at least one microbial kinase,
      whereby said system is capable of producing one or more phosphorylated recombinant caseins.

In a second aspect the present disclosure concerns a system for producing one or more phosphomimetic recombinant caseins, said system comprising a microbial host cell expressing:
i. at least one recombinant casein selected from the group consisting of alpha-casein S1 (αS1), alpha-casein S2 (αS2), beta-casein and kappa-casein, wherein one or more amino acid residues that are phosphorylated in a native mammalian casein are substituted with one or more phosphomimetic amino acids; and
ii. optionally at least one microbial kinase capable of phosphorylating said phosphomimetic casein at residues which are phosphorylated in a native mammalian casein, and which have not been substituted with phosphomimetic amino acids,
   whereby said system is capable of producing one or more phosphomimetic recombinant caseins.

In a third aspect the present disclosure concerns phosphorylated, recombinant casein.

In a fourth aspect the present disclosure concerns phosphomimetic recombinant casein.

In a fifth aspect the present disclosure concerns phosphorylated, phosphomimetic recombinant casein.

In a sixth aspect the present disclosure concerns a micelle comprising phosphorylated and/or phosphomimetic recombinant casein.

In a seventh aspect the present disclosure concerns a food product comprising phosphorylated and/or phosphomimetic recombinant casein.

### DESCRIPTION OF DRAWINGS

FIG. 1. Expression of Human Fam20C and Bovine Fam20C in E. coli. The proteins are expressed as aggregates in the insoluble fraction. A. Insoluble fraction. B. Soluble fraction. V: Vector; αCN: αₛ₁ CN; Hf: Human Fam20C; Bf: Bovine Fam20C domain; Bdp: Bovine Fam20C whole gene; M: Marker.

FIG. 2. Co-expression of casein α_{S1} and β, and PrkD in E. coli. The arrows indicate PrkD and the caseins α_{S1} and β.

FIG. 3. Co-expression of casein α_{S1} and YabT in E. coli. The arrows indicate YabT and α_{S1} and β caseins.

FIG. 4. Phosphomimetic variants of αₛ₁ and β casein having serine replaced with aspartic acid.

FIG. 5. Free calcium ions present in the sample after calcium binding assay.

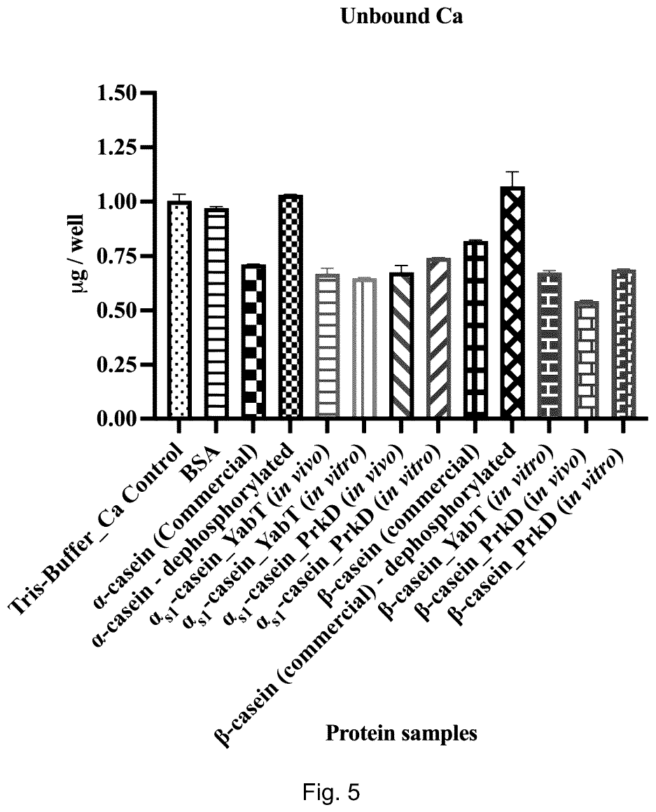

FIG. 6. Clustal O (1.2.4) multiple sequence alignment of alpha s1 casein from camel, human, goat, sheep, bovine and buffalo. Amino acids in bold(S) are phosphorylated.

FIG. 7. Clustal O (1.2.4) multiple sequence alignment of alpha s2 casein from camel, human, goat, sheep, bovine and buffalo. Amino acids in bold(S) are phosphorylated.

FIG. 8. Clustal O (1.2.4) multiple sequence alignment of beta casein from camel, human, goat, sheep, bovine and buffalo. Amino acids in bold (S, T) are phosphorylated.

FIG. 9. Clustal O (1.2.4) multiple sequence alignment of kappa casein from camel, human, goat, sheep, bovine and buffalo. Amino acids in bold (S, T) are phosphorylated.

### DETAILED DESCRIPTION

### Definitions

The term ‘milk protein’ as used herein refers to whey protein and casein protein, which are two types of protein found in milk, such as in milk from cows, goats, sheep, horse, camel, deer, reindeer and buffalo, humans, or other mammals.

The term ‘whey protein’ refers generally to a group of milk proteins that remain soluble when liquid milk is acidified to a pH of 4.6 or lower, whereas ‘casein proteins’ are milk proteins that coagulate at acidic pH to become cheese, yogurt, or another solidified or semi-solidified milk product.

The term ‘casein’ as used herein refers to alpha S1casein, alpha S2-casein beta casein or kappa casein (αS1, αS2, β-casein and κ-casein) which can be differentiated from one another by their amino acids composition, their charge distribution and their tendency to form aggregates in the presence of calcium.

The term ‘native casein’ as well as the term ‘native mammalian casein’ are used interchangeably, and refers to casein which can be obtained from mammalian milk.

The term ‘recombinant casein’ as used herein refers to casein produced by recombinant techniques, such as by microbial host cells. For example, a recombinant casein may be a mammalian (human, bovine or other) casein recombinantly expressed in a microbial host cell. The term ‘phosphorylated recombinant casein’ as used herein refers to recombinant casein which has been phosphorylated at one or more specific, native phosphorylation sites. The term ‘phosphomimetic recombinant casein’ as used herein refers to recombinant casein wherein specific native phosphorylation sites have been replaced with phosphomimetic amino acid. The term ‘phosphorylated, phosphomimetic recombinant casein’ refers to phosphomimetic casein, which has been phosphorylated at specific, native phosphorylation sites.

The term ‘in vivo phosphorylation’ as used herein refers to methods where one or more kinase genes and one or more casein genes are co-expressed in the same host cell, and where the expressed kinase(s) phosphorylates the expressed casein(s) either inside the host cell, or in the medium after secretion of the kinase(s) and casein(s).

The term ‘in vitro phosphorylation’ as used herein refers to methods where one or more casein genes are expressed in one host cell, and where the recombinantly produced caseins are phosphorylated outside the host cell after being put in contact with a kinase.

The term ‘co-expression’ as used herein should be understood as a simultaneous expression of casein and kinase, either in the same host cell or in different host cells in the same medium.

The term ‘host cell’ as used herein, refers to an organism selected from the group consisting of bacteria, yeast and filamentous fungi. Preferred bacteria are selected from the group consisting of lactic acid bacteria, for example Lactococcus spp. and Lactobacillus spp.; Corynebacterium spp., Pseudomonas spp., Streptomyces spp. Escherichia coli and Bacillus spp., for example Bacillus subtilis, Bacillus thuringiensis and Bacillus licheniformis. Preferred fungi are Trichoderma reesei, Aspergillus vadensis, Aspergillus oryzae and Aspergillus niger and preferred yeast cells are selected from the group consisting of Kluyveromyces spp., Pichia spp., Saccharomyces spp., Tetrahymena spp., Yarrowia spp., Hansenula spp., Blastobotrys spp., Aspergillus spp., Candida spp., Zygosaccharomyces spp., Trichoderma spp., Chrysosporium spp., Fusarium spp., and Neurospora spp. and Debaryomyces sp.

The term ‘kinase’ as used herein refers to class of enzymes which catalyse transfer of γ-phosphate from ATP to a hydroxyl group on a side chain of Ser/Thr or Tyr in proteins and peptides. The term ‘microbial kinase’ as used herein refers to kinases of microbial origin, such as bacterial kinases and/or fungal kinases.

The term ‘sequence identity’ as used herein refers to the percentage of residue matches between at least two polypeptide sequences aligned using a standardized algorithm. Such an algorithm may insert, in a standardized and reproducible way, gaps in the sequences being compared in order to optimize alignment between two sequences, and therefore achieve a more meaningful comparison of the two sequences. For purposes of the present invention, the sequence identity between two amino acid sequences is determined using the NCBI BLAST program.

The term ‘phosphomimetic amino acid’ as used herein refers to non-phosphorylated amino acids which mimic phosphorylated amino acids, without being phosphorylated. Those non-phosphorylated amino acids appear chemically similar to phosphorylated amino acids. As an example, aspartic acid (D) is chemically similar to phospho-serine (both carries negative charges at physiological pH). The replacement of a phosphor-serine with e.g. aspartic acid, is refers to as a phosphomimetic substitution. The substitution has the effect, that the phosphomimetic amino acid always will appear in its phosphorylated form, and thus mimic a phosphorylated amino acid.

The term ‘phosphomimetic protein’ as used herein refers to proteins in with amino acids which are phosphorylated in the native protein, referred to herein as ‘native phosphorylation sites’, are substituted with phosphomimetic amino acids. A phosphomimetic protein could be a casein, where a serine and/or a threonine, which is commonly phosphorylated, is substituted with a phosphomimetic amino acid, e.g., an aspartic acid (D), or a glutamic acid (E).

The term ‘degree of phosphorylation’ as used herein refers to the efficiency of the phosphorylation process performed by the kinase. A degree of phosphorylation of 80% at a native phosphorylation site X, means that 80% of the recombinant casein proteins are phosphorylated at the given native site X.

The term ‘functional recombinant casein’ as used herein refers to phosphorylated recombinant casein, phosphomimetic recombinant casein and phosphorylated, phosphomimetic recombinant casein, which has functional properties which allows for the use of the recombinant casein as a substitute for native casein. Examples of functional properties include calcium-binding properties, which is of importance for the formation of micelles.

### Casein

Casein has a wide variety of uses, being a major component of food products such as cheese and yoghurt, as well as being a valuable food additive due to its emulsifying properties.

In milk, native caseins form large colloidal particles between 50 to 600 nm in diameter (approximately 150 nm on average) referred to as casein micelles. The micelles form a very stable colloidal system in milk, being one of the main causes for its colour, heat stability and coagulation by acid and/or rennin

Phosphorylation plays an important role in the formation of casein micelles as the micelles are formed by calcium phosphate complexation by phosphoserine residues present in the casein structure. Phosphorylation of caseins enhances their ability to form and stabilize the casein micelle structure, which is essential for the texture, viscosity, and stability of dairy products like cheese and yogurt. Moreover the phosphorylation is important for gelling and emulsification properties.

When preparing recombinant casein, it is highly important that the caseins are phosphorylated at the corrects sites, with an appropriate degree of phosphorylation, as this is crucial for the structural and functional properties of the protein, e.g. for recombinant casein to be able to efficiently form micelles.

### Native Casein Phosphorylation Sites

Native mammalian caseins, including alpha S1 casein, alpha s2 casein, beta casein and kappa casein, are typically synthesized and phosphorylated in the lactating mammary gland. The native phosphorylation sites of caseins, are typically phosphorylated after translation by casein kinases, such as casein kinase 1 (CK1) and Fam20C.

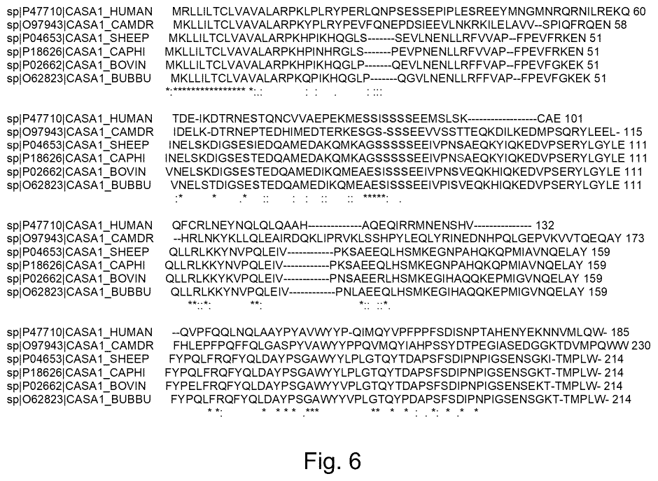

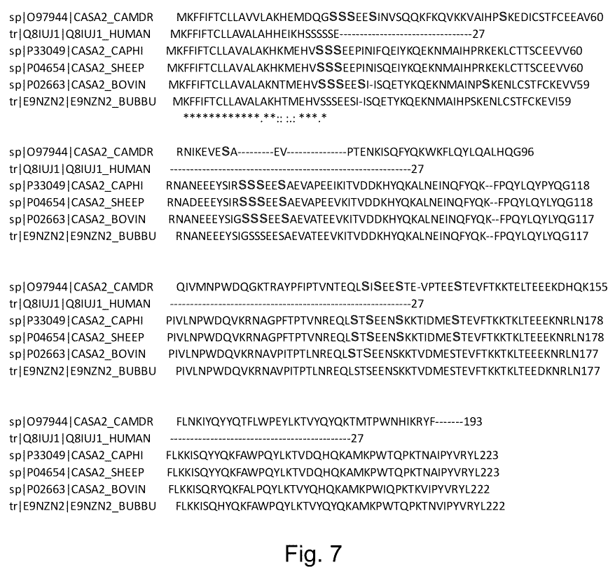

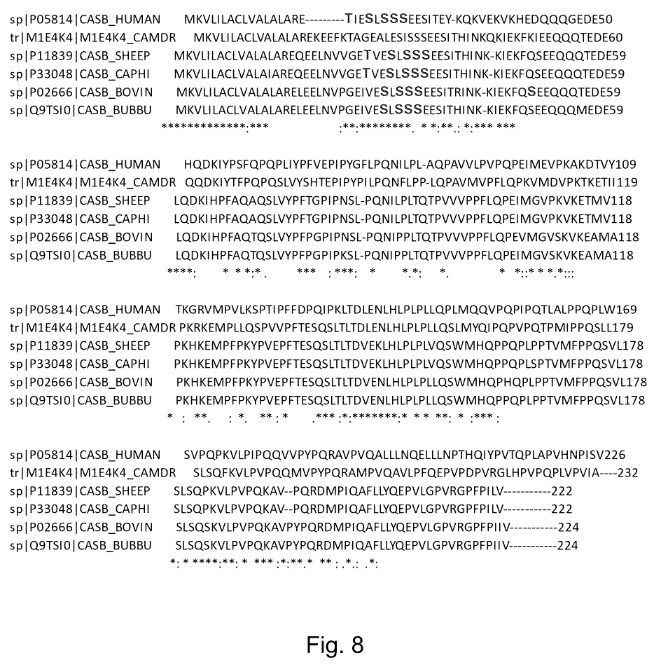

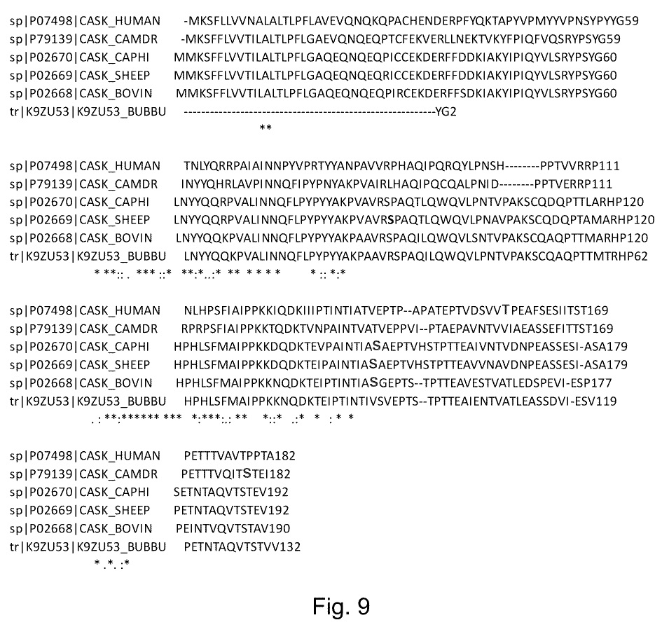

Native caseins produced by mammals typically present one or more native phosphorylation sites including both serine and threonine residues, which are well conserved between the different mammals as depicted in FIG. 6 (Alpha s1 casein, sequence alignment for camel, human, goat, sheep, bovine and buffalo), FIG. 7 (Alpha s2 casein, sequence alignment for camel, human, goat, sheep, bovine and buffalo), FIG. 8 (Beta casein, sequence alignment for camel, human, goat, sheep, bovine and buffalo), and FIG. 9 (Kappa casein, sequence alignment for camel, human, goat, sheep, bovine and buffalo).

Human alpha-s1 casein (SEQ ID NO: 10) is known to have the following native phosphorylation sites: S31, S33, S41, S71, S85, S86, S88, S89, S90, S91.

Camel alpha-s1 casein (SEQ ID NO: 11) is known to have the following native phosphorylation sites: S33, S83, S85, S86, S87, S88.

Sheep alpha-s1 casein (SEQ ID NO: 12) is known to have the following native phosphorylation sites: S27, S56, S61, S63, S79, S80, S81, S82, S83, S90, S130.

Goat alpha-s1 casein (SEQ ID NO: 13) is known to have the following native phosphorylation sites: S61, S63, S79, S80, S81, S82, S83, S90, S130.

Bovine alpha-s1 casein (SEQ ID NO: 14) is known to have the following native phosphorylation sites: S56, S61, S63, S79, S81, S82, S83, S90.

Buffalo alpha-s1 casein (SEQ ID NO: 15) is known to have the following native phosphorylation sites: S63, S79, S80, S81, S82, S83, S90.

Camel alpha-s2 casein (SEQ ID NO: 17) is known to have the following native phosphorylation sites: S23, S24, S25, S28, S47, S68, S123, S125, S128, S136.

Sheep alpha-s2 casein (SEQ ID NO: 18) is known to have the following native phosphorylation sites: S23, S24, S25, S72, S73, S74, S77, S145, S147, S151, S159.

Goat alpha-s2 casein (SEQ ID NO: 19) is known to have the following native phosphorylation sites: S23, S24, S25, S72, S73, S74, S77, S145, S147, S151, S159.

Bovine alpha-s2 casein (SEQ ID NO: 20) is known to have the following native phosphorylation sites: S23, S24, S25, S28, S46, S71, S72, S73, S76, S144, S146, S150, S158.

Buffalo alpha-s2 casein (SEQ ID NO: 21) is known to have the following native phosphorylation sites:

Human beta-casein (SEQ ID NO: 22) is known to have the following native phosphorylation sites: T18, S21, S23, S24, S25.

Camel beta-casein (SEQ ID NO: 23) is not known to have native phosphorylation sites.

Sheep beta-casein (SEQ ID NO: 24) is known to have the following native phosphorylation sites: T27, S30, S32, S33, S34.

Goat beta-casein (SEQ ID NO: 25) is known to have the following native phosphorylation sites: T27, S30, S32, S33, S34.

Bovine beta-casein (SEQ ID NO: 26) is known to have the following native phosphorylation sites: S30, S32, S33, S34, S50.

Buffalo beta-casein (SEQ ID NO: 27) is known to have the following native phosphorylation sites: S30, S32, S33, S34.

Human kappa-casein (SEQ ID NO: 28) is known to have the following native phosphorylation sites: T157.

Camel kappa-casein (SEQ ID NO: 29) is not known to have native phosphorylation sites.

Sheep kappa-casein (SEQ ID NO: 30) is known to have the following native phosphorylation sites: S148, S172, S189.

Goat kappa-casein (SEQ ID NO: 31) is known to have the following native phosphorylation sites: S148, T166, S170, S187.

Bovine kappa-casein (SEQ ID NO: 32) is known to have the following native phosphorylation sites: S148, T166, S170, S187.

Buffalo kappa-casein (SEQ ID NO: 33) is not known to have native phosphorylation sites.

### Systems and Methods for Production of Recombinant Casein

The current disclosure relates to different strategies to produce recombinant caseins in microorganisms such as bacteria, yeasts, fungi.

In one embodiment, the system the system of the current disclosure relates to recombinant casein, wherein the recombinant casein is a recombinant variant of a native mammalian casein, optionally wherein said mammalian casein is that found in milk from a mammal selected from the group consisting of human, cow, buffalo, zebu, yak, equines, sheep, goat, horse, donkey, camel, deer, and reindeer.

In one embodiment the native mammalian casein is a casein natively produced in a mammal, such as a casein natively produced in human, cow, buffalo, zebu, yak, equines, sheep, goat, horse, donkey, camel, deer, and reindeer, and wherein said native mammalian casein is naturally phosphorylated at one or more native phosphorylation sites.

In one embodiment the recombinant casein is recombinant bovine casein.

In one embodiment the recombinant bovine casein is recombinant alpha-casein (αS1, αS2), beta-casein and/or kappa-casein.

In one embodiment the recombinant casein is recombinant human casein.

In one embodiment the recombinant casein is recombinant human alpha-casein (αS1, αS2), beta-casein or kappa-casein.

In one embodiment the recombinant casein of the current disclosure is a casein selected from the group consisting of: SEQ ID NO: 1, SEQ ID NO: 3, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33 or a variant thereof, wherein said variant has at least 70% sequence identity, such as at least 75% sequence identity, such as at least 80% sequence identity, such as at least 85% sequence identity, such as at least 88% sequence identity, such as at least 90% sequence identity, such as at least 95% sequence identity, such as at least 96% sequence identity, such as at least 97% sequence identity, such as at least 98% sequence identity, such as at least 99% sequence identity, to any one of SEQ ID NO: 1, SEQ ID NO: 3, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33.

### Phosphorylated Recombinant Casein

For recombinant casein to become an alternative to casein obtained from mammalian milk, it is important that the recombinant casein holds at least some of the functional properties of casein. The phosphorylation pattern of casein plays an important role in the formation of casein micelles, as the micelle formation is supported by complexation between phosphoserine residues and calcium. Thus it is important that the recombinant casein is sufficiently phosphorylated, both in terms of phosphorylated sites, and degree of phosphorylation at the phosphorylated sites.

The present disclosure presents methods for production of phosphorylated recombinant casein. The present disclosure moreover presents methods for production of phosphomimetic casein, and methods for production of phosphorylated, phosphomimetic recombinant casein.

In one embodiment the current disclosure relates to a system for producing one or more phosphorylated recombinant caseins.

In one embodiment the system comprises:
i. a microbial host cell expressing:
   a. at least one recombinant casein selected from the group consisting of alpha-casein αS1, alpha-casein αS2, beta-casein and kappa-casein and
   b. at least one microbial kinase;
   or
ii. a microbial host cell expressing at least one casein selected from the group consisting of alpha-casein αS1, alpha-casein αS2, beta-casein or kappa-casein, and
   a composition comprising at least one microbial kinase,
   whereby said microbial host cell is capable of producing one or more phosphorylated recombinant casein.

In one embodiment the present disclosure relates to a method of producing recombinant casein in a microbial host cell, comprising the steps of:
i. Providing a microbial host cell according to the present disclosure,
ii. incubating said microbial host cell in a medium, under conditions allowing growth of the microbial host cell, whereby at least one recombinant casein is produced; and
iii. incubating the at least one casein with at least one microbial kinase,
   whereby phosphorylated recombinant casein is produced.

In one embodiment the host cell according to the present disclosure is engineered to express a casein gene, such as a mammalian casein gene, wherein said gene is inserted into an expression vector and transformed into the host cell.

In one embodiment the kinase is capable of phosphorylating one or more amino acid residues of said recombinant casein at its native phosphorylation sites.

In one embodiment, the method of producing recombinant casein, comprises a step of recovering the recombinant casein.

In one embodiment the recombinant casein is phosphorylated at one or more native phosphorylation sites. In one embodiment the recombinant casein is phosphorylated at two native phosphorylation sites. In one embodiment the recombinant casein is phosphorylated at three native phosphorylation sites. In one embodiment the recombinant casein is phosphorylated at four native phosphorylation sites. In one embodiment the recombinant casein is phosphorylated at five native phosphorylation sites. In one embodiment the recombinant casein is phosphorylated at six native phosphorylation sites. In one embodiment the recombinant casein is phosphorylated at seven native phosphorylation sites. In one embodiment the recombinant casein is phosphorylated at eight native phosphorylation sites. In one embodiment the recombinant casein is phosphorylated at nine native phosphorylation sites. In one embodiment the recombinant casein is phosphorylated at ten native phosphorylation sites. In one embodiment the recombinant casein is phosphorylated at more than ten native phosphorylation sites.

In one embodiment the recombinant casein is phosphorylated at all its native phosphorylation sites. Native phosphorylation sites are serine and/or threonine residues and are described in detail herein in a dedicated section.

In one embodiment one or more serine and/or threonine residues of the recombinant casein, such as of a casein selected from the group consisting of:
alpha casein (αS1) as set forth in SEQ ID NO 10, 11, 12, 13 14 and 15, or a variant thereof, wherein said variant has at least 70% sequence identity to any one of SEQ ID NO 10, 11, 12, 13 14 and 15;
alpha casein (αS2) as set forth in SEQ ID NO 16, 17, 18, 19, 20 and 21, or a variant thereof, wherein said variant has at least 70% sequence identity to any one of SEQ ID NO 16, 17, 18, 19, 20 and 21;
beta casein as set forth in SEQ ID NO 22, 23, 24, 25, 26, and 27, or a variant thereof, wherein said variant has at least 70% sequence identity to any one of SEQ ID NO 22, 23, 24, 25, 26, and 27; and
kappa casein as set forth in SEQ ID NO 28, 29, 30, 31, 32 and 33, or a variant thereof, wherein said variant has at least 70% sequence identity to any one of SEQ ID NO 28, 29, 30, 31, 32 and 33,
are phosphorylated.

In one embodiment the recombinant bovine alpha casein (αS1) as set forth in SEQ ID NO: 1, or a variant thereof having at least 70% sequence identity to in SEQ ID NO: 1, is phosphorylated at least at one of the following positions: S46, S48, S64, S66, S67, S68, and S75.

In one embodiment the recombinant bovine alpha-S1 casein as set forth in SEQ ID NO: 14, or a variant thereof having at least 70% sequence identity to in SEQ ID NO: 14, is phosphorylated at least at one of the following positions: S56, S61, S63, S79, S81, S82, S83, and S90.

In one embodiment the recombinant bovine alpha-S2 casein as set forth in SEQ ID NO: 20, or a variant thereof having at least 70% sequence identity to in SEQ ID NO: 20, is phosphorylated at least at one of the following positions: S23, S24, S25, S28, S46, S71, S72, S73, S76, S144, S146, S150, or S158.

In one embodiment the recombinant bovine beta casein as set forth in SEQ ID NO: 3, or a variant thereof having at least 70% sequence identity to in SEQ ID NO: 3, is phosphorylated at least at one of the following positions: S15, S17, S18, S19 and S35.

In one embodiment the recombinant bovine beta casein as set forth in SEQ ID NO: 26, or a variant thereof having at least 70% sequence identity to in SEQ ID NO: 26, is phosphorylated at least at one of the following positions: S30, S32, S33, S34 and S50.

In one embodiment the recombinant bovine kappa casein as set forth in SEQ ID NO: 32, or a variant thereof having at least 70% sequence identity to in SEQ ID NO: 32, is phosphorylated at least at one of the following positions: S148, T166, S170, or S187.

In one embodiment the recombinant human alpha-S1 casein as set forth in SEQ ID NO: 10, or a variant thereof having at least 70% sequence identity to in SEQ ID NO: 10, is phosphorylated at least at one of the following positions: S31, S33, S41, S71, S85, S86, S88, S89, S90, or S91.

In one embodiment the recombinant human beta casein as set forth in SEQ ID NO: 22, or a variant thereof having at least 70% sequence identity to in SEQ ID NO: 22, is phosphorylated at least at one of the following positions: T18, S21, S23, S24, or S25.

In one embodiment the recombinant human kappa casein as set forth in SEQ ID NO: 28 or a variant thereof having at least 70% sequence identity to in SEQ ID NO: 28, is phosphorylated at least at one of the following positions: T157.

In one embodiment the phosphorylated recombinant casein of the current disclosure is a casein having a sequence selected from the group consisting of: SEQ ID NO: 1, SEQ ID NO: 3, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a variant thereof, wherein said variant has at least 70% sequence identity to any one of SEQ ID NO: 1, SEQ ID NO: 3, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, and wherein one or more amino acid residues of said casein or variant thereof are phosphorylated at:
one or more positions selected from the group consisting of S46, S48, S64, S66, S67, S68, and S75 of SEQ ID NO: 1;
one or more positions selected from the group consisting of S15, S17, S18, S19 and S35 of SEQ ID NO: 3;
one or more positions selected from the group consisting of S31, S33, S41, S71, S85, S86, S88, S89, S90, and S91 of SEQ ID NO: 10;
one or more positions selected from the group consisting of S33, S83, S85, S86, S87, and S88 of SEQ ID NO: 11;
one or more positions selected from the group consisting of S27, S56, S61, S63, S79, S80, S81, S82, S83, S90, and S130 of SEQ ID NO: 12;
one or more positions selected from the group consisting of S61, S63, S79, S80, S81, S82, S83, S90, and S130 of SEQ ID NO: 13;
one or more positions selected from the group consisting of S56, S61, S63, S79, S81, S82, S83, and S90 of SEQ ID NO: 14;
one or more positions selected from the group consisting of S63, S79, S80, S81, S82, S83, and S90 of SEQ ID NO: 15;
one or more positions selected from the group consisting of S23, S24, S25, S28, S47, S68, S123, S125, S128, and S136 of SEQ ID NO: 17;
one or more positions selected from the group consisting of S23, S24, S25, S72, S73, S74, S77, S145, S147, S151, and S159 of SEQ ID NO: 18;
one or more positions selected from the group consisting of S23, S24, S25, S72, S73, S74, S77, S145, S147, S151, and S159 of SEQ ID NO: 19;
one or more positions selected from the group consisting of S23, S24, S25, S28, S46, S71, S72, S73, S76, S144, S146, S150, and S158 of SEQ ID NO:20;
one or more positions selected from the group consisting of T18, S21, S23, S24, and S25 of SEQ ID NO:22;
one or more positions selected from the group consisting of T27, S30, S32, S33, and S34 of SEQ ID NO:24;
one or more positions selected from the group consisting of T27, S30, S32, S33, and S34 of SEQ ID NO:25;
one or more positions selected from the group consisting of S30, S32, S33, S34 and S50 of SEQ ID NO:26;
one or more positions selected from the group consisting of S30, S32, S33, and S34 of SEQ ID NO:27;
or position T157 of SEQ ID NO:28;
one or more positions selected from the group consisting of S148, S172, and S189 SEQ ID NO: 30;
one or more positions selected from the group consisting of S148, T166, S170, and S187 of SEQ ID NO:31;
one or more positions selected from the group consisting of S148, T166, S170, and S187 of SEQ ID NO:32.

The degree of phosphorylation can be determined using MS (Beausoleil, et al, 2006).

In one embodiment the phosphorylated recombinant casein has a degree of phosphorylation at each native phosphorylation site of at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as about 100%.

### Phosphomimetic Recombinant Casein

Phosphorylation as presented above, introduces negative charges to the phosphorylated amino acid residues. Another feasible way of introducing negative charges to casein, is the introduction of phosphomimetic amino acids, and thus the preparation of phosphomimetic recombinant casein.

The current disclosure presents methods for production of recombinant casein comprising phosphomimetic amino acids.

In one embodiment the current disclosure relates to a system for producing one or more phosphomimetic recombinant caseins. In one embodiment the system comprises a microbial host cell expressing:
i. at least one recombinant casein selected from the group consisting of alpha-casein (αS1), alpha-casein (αS2), beta-casein and kappa-casein, wherein one or more amino acid residues that are phosphorylated in a native mammalian casein are substituted with one or more phosphomimetic amino acids; and
ii. optionally at least one microbial kinase capable of phosphorylating said phosphomimetic casein at residues which are phosphorylated in a native mammalian casein, and which have not been substituted with phosphomimetic amino acids,
   whereby said host cell is capable of producing one or more phosphomimetic recombinant caseins.

In one embodiment one or more amino acids have been substituted with a phosphomimetic amino acid.

In one embodiment one or more serine and/or threonine residues have been substituted with a phosphomimetic amino acid.

In one embodiment one or more amino acid residues which are phosphorylated in the native mammalian casein have been substituted with one or more phosphomimetic amino acids.

In one embodiment the phosphomimetic amino acid is a natural amino acid. In one embodiment the phosphomimetic amino acid is an unnatural amino acid.

In one embodiment the phosphomimetic amino acid carries negatively charged functional groups.

In one embodiment the phosphomimetic amino acid carries carboxylic acid groups.

In one embodiment the phosphomimetic amino acid is independently selected from the group consisting of aspartic acid, glutamic acid and/or combinations thereof.

In one embodiment one or more serine and/or threonine residues of the recombinant casein, such as of:
alpha casein (αS1) as set forth in any one of SEQ ID NOs: 1, 10, 11, 12, 13, 14 and 15,
alpha casein (αS2) as set forth in any one of SEQ ID NOs: 16, 17, 18, 19, 20 and 21, beta casein as set forth in any one of SEQ ID NOs: 3, 22, 23, 24, 25, 26, and 27, and kappa casein as set forth in any one of SEQ ID NOs: 28, 29, 30, 31, 32 and 33,
have been substituted with one or more phosphomimetic amino acids.

In one embodiment the phosphomimetic recombinant casein of the current disclosure is a casein having a sequence selected from the group consisting of: SEQ ID NO: 1, SEQ ID NO: 3, of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a variant thereof, wherein said variant has at least 70% sequence identity to any one of SEQ ID NO: 1, SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, and wherein one or more amino acid residues of said variant are individually substituted by a phosphomimetic amino acids, wherein the substituted amino acid residues correspond to:
one or more positions selected from the group consisting of S46, S48, S64, S66, S67, S68, and S75 of SEQ ID NO: 1;
one or more positions selected from the group consisting of S15, S17, S18, S19 and S35 of SEQ ID NO: 3;
one or more positions selected from the group consisting of S31, S33, S41, S71, S85, S86, S88, S89, S90, and S91 of SEQ ID NO: 10;
one or more positions selected from the group consisting of S33, S83, S85, S86, S87, and S88 of SEQ ID NO: 11;
one or more positions selected from the group consisting of S27, S56, S61, S63, S79, S80, S81, S82, S83, S90, and S130 of SEQ ID NO: 12;
one or more positions selected from the group consisting of S61, S63, S79, S80, S81, S82, S83, S90, and S130 of SEQ ID NO: 13;
one or more positions selected from the group consisting of S56, S61, S63, S79, S81, S82, S83, and S90 of SEQ ID NO: 14;
one or more positions selected from the group consisting of S63, S79, S80, S81, S82, S83, and S90 of SEQ ID NO: 15;
one or more positions selected from the group consisting of S23, S24, S25, S28, S47, S68, S123, S125, S128, and S136 of SEQ ID NO: 17;
one or more positions selected from the group consisting of S23, S24, S25, S72, S73, S74, S77, S145, S147, S151, and S159 of SEQ ID NO: 18;
one or more positions selected from the group consisting of S23, S24, S25, S72, S73, S74, S77, S145, S147, S151, and S159 of SEQ ID NO: 19;
one or more positions selected from the group consisting of S23, S24, S25, S28, S46, S71, S72, S73, S76, S144, S146, S150, and S158 of SEQ ID NO: 20;
one or more positions selected from the group consisting of T18, S21, S23, S24, and S25 of SEQ ID NO: 22;
one or more positions selected from the group consisting of T27, S30, S32, S33, and S34 of SEQ ID NO: 24;
one or more positions selected from the group consisting of T27, S30, S32, S33, and S34 of SEQ ID NO: 25;
one or more positions selected from the group consisting of S30, S32, S33, S34 and S50 of SEQ ID NO: 26;
one or more positions selected from the group consisting of S30, S32, S33, and S34 of SEQ ID NO: 27;
or position T157 of SEQ ID NO: 28;
one or more positions selected from the group consisting of S148, S172, and S189 SEQ ID NO: 30;
one or more positions selected from the group consisting of S148, T166, S170, and S187 of SEQ ID NO: 31;
one or more positions selected from the group consisting of S148, T166, S170, and S187 of SEQ ID NO: 32.

In one embodiment the recombinant bovine alpha casein (αS1) as set forth in SEQ ID NO: 1, or a variant thereof having at least 70% sequence identity to SEQ ID NO: 1, comprises a phosphomimetic amino acid at least at one of the following positions: S46, S48, S64, S66, S67, S68, and S75.

In one embodiment the recombinant bovine alpha-S1 casein as set forth in SEQ ID NO: 14, or a variant thereof having at least 70% sequence identity to SEQ ID NO: 14, comprises a phosphomimetic amino acid at least at one of the following positions: S56, S61, S63, S79, S81, S82, S83, or S90.

In one embodiment the recombinant bovine alpha-S2 casein as set forth in SEQ ID NO: 20, or a variant thereof having at least 70% sequence identity to SEQ ID NO: 20, comprises a phosphomimetic amino acid at least at one of the following positions: S23, S24, S25, S28, S46, S71, S72, S73, S76, S144, S146, S150, or S158.

In one embodiment the recombinant bovine beta casein as set forth in SEQ ID NO: 3, or a variant thereof having at least 70% sequence identity to SEQ ID NO: 3, comprises a phosphomimetic amino acid at least at one of the following positions: S15, S17, S18, S19 and S35.

In one embodiment the recombinant bovine beta casein as set forth in SEQ ID NO: 26, or a variant thereof having at least 70% sequence identity to SEQ ID NO: 26, comprises a phosphomimetic amino acid at least at one of the following positions: S30, S32, S33, S34 and S50.

In one embodiment the recombinant bovine kappa casein as set forth in SEQ ID NO: 32, or a variant thereof having at least 70% sequence identity to SEQ ID NO: 32, comprises a phosphomimetic amino acid at least at one of the following positions: S148, T166, S170, or S187.

In one embodiment the recombinant human alpha-S1 casein as set forth in SEQ ID NO: 10, or a variant thereof having at least 70% sequence identity to SEQ ID NO: 10, comprises a phosphomimetic amino acid at least at one of the following positions: S31, S33, S41, S71, S85, S86, S88, S89, S90, or S91.

In one embodiment the recombinant human beta casein as set forth in SEQ ID NO: 22, or a variant thereof having at least 70% sequence identity to SEQ ID NO: 22, comprises a phosphomimetic amino acid at least at one of the following positions: T18, S21, S23, S24, or S25.

In one embodiment the recombinant human kappa casein as set forth in SEQ ID NO: 28, or a variant thereof having at least 70% sequence identity to SEQ ID NO: 28, comprises a phosphomimetic amino acid at least at one of the following positions: T157.

In one embodiment the phosphomimetic, recombinant casein comprises one phosphomimetic amino acid. In one embodiment the phosphomimetic, recombinant casein comprises two phosphomimetic amino acids. In one embodiment the phosphomimetic, recombinant casein comprises three phosphomimetic amino acids. In one embodiment the phosphomimetic, recombinant casein comprises four phosphomimetic amino acids. In one embodiment the phosphomimetic, recombinant casein comprises five phosphomimetic amino acids. In one embodiment the phosphomimetic, recombinant casein comprises six phosphomimetic amino acids. In one embodiment the phosphomimetic, recombinant casein comprises seven phosphomimetic amino acids. In one embodiment the phosphomimetic, recombinant casein comprises eight phosphomimetic amino acids. In one embodiment the phosphomimetic, recombinant casein comprises nine phosphomimetic amino acids. In one embodiment the phosphomimetic, recombinant casein comprises ten phosphomimetic amino acids. In one embodiment the phosphomimetic, recombinant casein comprises eleven phosphomimetic amino acids. In one embodiment the phosphomimetic, recombinant casein comprises twelve phosphomimetic amino acids. In one embodiment the phosphomimetic, recombinant casein comprises thirteen phosphomimetic amino acids. In one embodiment the phosphomimetic, recombinant casein comprises fourteen phosphomimetic amino acids. In one embodiment the phosphomimetic, recombinant casein comprises fifteen phosphomimetic amino acids.

In one embodiment the present disclosure relates to a method of producing recombinant casein in a microbial host cell, comprising the steps of:
i. Providing a microbial host cell encoding a recombinant casein as described in the present disclosure; and
ii. incubating said microbial host cell in a medium, under conditions allowing growth of the host cell of at least one phosphomimetic casein,
   whereby at least one phosphomimetic recombinant casein is produced.

In one embodiment of the present disclosure, the one or more phosphomimetic recombinant casein is as set forth in SEQ ID NO: 2 or SEQ ID NO. 4, or a variant thereof having at least 90% sequence identity, similarity or homology thereto.

### Phosphorylated, Phosphomimetic Recombinant Casein

The current disclosure presents methods for production of phosphorylated recombinant casein comprising phosphomimetic amino acids.

In one embodiment the phosphorylated, phosphomimetic recombinant casein has a degree of phosphorylation at each phosphorylated native phosphorylation site of at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as about 100%.

In one embodiment the phosphomimetic, phosphorylated recombinant casein of the current disclosure is a casein having a sequence selected from the group consisting of: SEQ ID NO: 1, SEQ ID NO: 3, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a variant thereof, wherein said variant has at least 70% sequence identity to any one of SEQ ID NO: 1, SEQ ID NO: 3, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, and wherein:
i) one or more amino acid residues of said casein or variant thereof are phosphorylated at;
one or more positions selected from the group consisting of S46, S48, S64, S66, S67, S68, and S75 of SEQ ID NO: 1;
one or more positions selected from the group consisting of S15, S17, S18, S19 and S35 of SEQ ID NO: 3;
one or more positions selected from the group consisting of S31, S33, S41, S71, S85, S86, S88, S89, S90, and S91 of SEQ ID NO: 10;
one or more positions selected from the group consisting of S33, S83, S85, S86, S87, and S88 of SEQ ID NO: 11;
one or more positions selected from the group consisting of S27, S56, S61, S63, S79, S80, S81, S82, S83, S90, and S130 of SEQ ID NO: 12;
one or more positions selected from the group consisting of S61, S63, S79, S80, S81, S82, S83, S90, and S130 of SEQ ID NO: 13;
one or more positions selected from the group consisting of S56, S61, S63, S79, S81, S82, S83, and S90 of SEQ ID NO: 14;
one or more positions selected from the group consisting of S63, S79, S80, S81, S82, S83, and S90 of SEQ ID NO: 15;
one or more positions selected from the group consisting of S23, S24, S25, S28, S47, S68, S123, S125, S128, and S136 of SEQ ID NO: 17;
one or more positions selected from the group consisting of S23, S24, S25, S72, S73, S74, S77, S145, S147, S151, and S159 of SEQ ID NO: 18;
one or more positions selected from the group consisting of S23, S24, S25, S72, S73, S74, S77, S145, S147, S151, and S159 of SEQ ID NO: 19;
one or more positions selected from the group consisting of S23, S24, S25, S28, S46, S71, S72, S73, S76, S144, S146, S150, and S158 of SEQ ID NO: 20;
one or more positions selected from the group consisting of T18, S21, S23, S24, and S25 of SEQ ID NO: 22;
one or more positions selected from the group consisting of T27, S30, S32, S33, and S34 of SEQ ID NO: 24;
one or more positions selected from the group consisting of T27, S30, S32, S33, and S34 of SEQ ID NO: 25;
one or more positions selected from the group consisting of S30, S32, S33, S34 and S50 of SEQ ID NO: 26;
one or more positions selected from the group consisting of S30, S32, S33, and S34 of SEQ ID NO: 27;
or position T157 of SEQ ID NO: 28;
one or more positions selected from the group consisting of S148, S172, and S189 SEQ ID NO: 30;
one or more positions selected from the group consisting of S148, T166, S170, and S187 of SEQ ID NO: 31;
one or more positions selected from the group consisting of S148, T166, S170, and S187 of SEQ ID NO: 32 and
ii) one or more amino acid residues of said variant are individually substituted by a phosphomimetic amino acids, wherein the substituted amino acid residues correspond to:
one or more positions selected from the group consisting of S46, S48, S64, S66, S67, S68, and S75 of SEQ ID NO: 1;
one or more positions selected from the group consisting of S15, S17, S18, S19 and S35 of SEQ ID NO: 3;
one or more positions selected from the group consisting of S31, S33, S41, S71, S85, S86, S88, S89, S90, and S91 of SEQ ID NO: 10;
one or more positions selected from the group consisting of S33, S83, S85, S86, S87, and S88 of SEQ ID NO: 11;
one or more positions selected from the group consisting of S27, S56, S61, S63, S79, S80, S81, S82, S83, S90, and S130 of SEQ ID NO: 12;
one or more positions selected from the group consisting of S61, S63, S79, S80, S81, S82, S83, S90, and S130 of SEQ ID NO: 13;
one or more positions selected from the group consisting of S56, S61, S63, S79, S81, S82, S83, and S90 of SEQ ID NO: 14;
one or more positions selected from the group consisting of S63, S79, S80, S81, S82, S83, and S90 of SEQ ID NO: 15;
one or more positions selected from the group consisting of S23, S24, S25, S28, S47, S68, S123, S125, S128, and S136 of SEQ ID NO: 17;
one or more positions selected from the group consisting of S23, S24, S25, S72, S73, S74, S77, S145, S147, S151, and S159 of SEQ ID NO: 18;
one or more positions selected from the group consisting of S23, S24, S25, S72, S73, S74, S77, S145, S147, S151, and S159 of SEQ ID NO: 19;
one or more positions selected from the group consisting of S23, S24, S25, S28, S46, S71, S72, S73, S76, S144, S146, S150, and S158 of SEQ ID NO: 20;
one or more positions selected from the group consisting of T18, S21, S23, S24, and S25 of SEQ ID NO: 22;
one or more positions selected from the group consisting of T27, S30, S32, S33, and S34 of SEQ ID NO: 24;
one or more positions selected from the group consisting of T27, S30, S32, S33, and S34 of SEQ ID NO: 25;
one or more positions selected from the group consisting of S30, S32, S33, S34 and S50 of SEQ ID NO: 26;
one or more positions selected from the group consisting of S30, S32, S33, and S34 of SEQ ID NO: 27;
or position T157 of SEQ ID NO: 28;
one or more positions selected from the group consisting of S148, S172, and S189 SEQ ID NO: 30;
one or more positions selected from the group consisting of S148, T166, S170, and S187 of SEQ ID NO: 31;
one or more positions selected from the group consisting of S148, T166, S170, and S187 of SEQ ID NO: 32.

In one embodiment the recombinant bovine alpha casein (αS1) as set forth in SEQ ID NO: 1, or a variant thereof having at least 70% sequence identity to SEQ ID NO: 1,
i) comprises a phosphomimetic amino acid at least at one position selected from the group consisting of: S46, S48, S64, S66, S67, S68, and S75, and
ii) is phosphorylated at least at one position selected from the group consisting of: S46, S48, S64, S66, S67, S68, and S75, which is not substituted by a phosphomimetic amino acid.

In one embodiment the recombinant bovine alpha-S1 casein as set forth in SEQ ID NO: 14, or a variant thereof having at least 70% sequence identity to SEQ ID NO: 14
i) comprises a phosphomimetic amino acid at least at one position selected from the group consisting of: S56, S61, S63, S79, S81, S82, S83, and S90, and
ii) is phosphorylated at least at one position selected from the group consisting of: S56, S61, S63, S79, S81, S82, S83, and S90, which is not substituted by a phosphomimetic amino acid.

In one embodiment the recombinant bovine alpha-S2 casein as set forth in SEQ ID NO: 20, or a variant thereof having at least 70% sequence identity to SEQ ID NO: 20
i) comprises a phosphomimetic amino acid at least at one position selected from the group consisting of: S23, S24, S25, S28, S46, S71, S72, S73, S76, S144, S146, S150, and S158, and
ii) is phosphorylated at least at one position selected from the group consisting of: S23, S24, S25, S28, S46, S71, S72, S73, S76, S144, S146, S150, and S158, which is not substituted by a phosphomimetic amino acid.

In one embodiment the recombinant bovine beta casein as set forth in SEQ ID NO: 3, or a variant thereof having at least 70% sequence identity to SEQ ID NO: 3
i) comprises a phosphomimetic amino acid at least at a position selected from the group consisting of: S15, S17, S18, S19 and S35, and
ii) is phosphorylated at least at one position selected from the group consisting of: S15, S17, S18, S19 and S35, which is not substituted by a phosphomimetic amino acid.

In one embodiment the recombinant bovine beta casein as set forth in SEQ ID NO: 26, or a variant thereof having at least 70% sequence identity to SEQ ID NO: 26
i) comprises a phosphomimetic amino acid at least at one position selected from the group consisting of: S30, S32, S33, S34 and S50, and
ii) is phosphorylated at least at one position selected from the group consisting of: S30, S32, S33, S34 and S50, which is not substituted by a phosphomimetic amino acid.

In one embodiment the recombinant bovine kappa casein as set forth in SEQ ID NO: 32, or a variant thereof having at least 70% sequence identity to SEQ ID NO: 32
i) comprises a phosphomimetic amino acid at least at one position selected from the group consisting of: S148, T166, S170, and S187, and
ii) is phosphorylated at least at one position selected from the group consisting of: S148, T166, S170, and S187, which is not substituted by a phosphomimetic amino acid.

In one embodiment the recombinant human alpha-S1 casein as set forth in SEQ ID NO: 10, or a variant thereof having at least 70% sequence identity to SEQ ID NO: 10
i) comprises a phosphomimetic amino acid at least at one position selected from the group consisting of: S31, S33, S41, S71, S85, S86, S88, S89, S90, and S91, and
ii) is phosphorylated at least at one position selected from the group consisting of: S31, S33, S41, S71, S85, S86, S88, S89, S90, and S91, which is not substituted by a phosphomimetic amino acid.

In one embodiment the recombinant human beta casein as set forth in SEQ ID NO: 22, or a variant thereof having at least 70% sequence identity to SEQ ID NO: 22
i) comprises a phosphomimetic amino acid at least at one position selected from the group consisting of: T18, S21, S23, S24, and S25, and
ii) is phosphorylated at least at one position selected from the group consisting of: T18, S21, S23, S24, and S25, which is not substituted by a phosphomimetic amino acid.

In one embodiment the present disclosure relates to a method of producing recombinant casein in a microbial host cell, comprising the steps of:
i. Providing a microbial host cell encoding a recombinant casein as described in the present disclosure; and
ii. incubating said microbial host cell in a medium, under conditions sufficient to allow for production of at least one phosphomimetic casein, whereby at least one phosphomimetic casein is produced;
iii. incubating the at least one casein with at least one microbial kinase,
   whereby phosphorylated and/or phosphomimetic recombinant casein is produced.

### Expression System

In one embodiment the present disclosure relates to an expression system for expression of phosphorylated recombinant casein in a microbial host cell, comprising:
i. a first nucleic acid encoding at least one recombinant casein selected from the group consisting of alpha S1 casein (αS1), alpha-S2 casein (αS2), beta-casein and kappa-casein; and
ii. a second nucleic acid encoding a kinase.

In one embodiment the nucleic acid encoding a casein and the nucleic acid encoding a kinase are expressed in a microbial host cell.

In one embodiment the nucleic acid encoding at least one casein is expressed in a first microbial host cell, and the nucleic acid encoding at least one kinase is expressed in a second microbial host cell.

In one embodiment the present disclosure relates to the use of an expression system as defined elsewhere in the description, for production of at least one recombinant casein of interest.

In one embodiment the at least one recombinant casein expressed by an expression system, is selected from the group consisting of SEQ ID NO: 1, SEQ ID NO: 3, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a variant thereof having at least 70%, such as at least 75%, such as at least 80%, such as at least 85%, such as at least 90%, such as at least 95%, such as at least 96%, such as at least 97%, such as at least 98%, such as at least 99%, sequence identity, similarity, or homology thereto.

### Host Cell

The present disclosure also relates to a microbial host cell genetically engineered to express at least one casein.

In one embodiment the microbial host cell is a bacterial cell, a fungal cell or a yeast cell. In one embodiment the microbial host cell comprises at least one protease deficiency rendering said microbial host cell completely or partially deficient in the production and/or function of one or more proteases.

In one embodiment the microbial host cell expresses at least one chaperone protein.

In one embodiment the present disclosure relates to a microbial host cell comprising a system as defined elsewhere in the description, wherein said host cell is capable of producing a recombinant casein.

In one embodiment the microbial host cell is a bacterial cell, which belongs to a genus selected from the group consisting of Bacillus spp., Escherichia spp., Corynebacterium spp., Pseudomonas spp., and Streptomyces spp. For example, the microbial host cell may belong to a species selected from Bacillus subtilis, Escherichia coli,

In one embodiment the microbial host cell is a bacterial cell, in particular a lactic acid bacteria cell, for example a bacterial cell belonging to Lactococcus spp. and Lactobacillus spp.

In one embodiment the microbial host cell is a fungal host cell, which belongs to a genus selected from the group consisting of Trichoderma spp., Aspergillus spp., Kluyveromyces spp., Pichia spp., Saccharomyces spp., Tetrahymena spp., Yarrowia spp., Hansenula spp., Blastobotrys spp., Candida spp., Zygosaccharomyces spp., Chrysosporium spp., Fusarium spp., Neurospora spp. and Debaryomyces spp.

In one embodiment the microbial host cell is a fungal host cell, which belongs to a species selected from selected from the group consisting Trichoderma reesei, Aspergillus vadensis, Aspergillus oryzae and Aspergillus niger.

### Kinase

In one embodiment the microbial kinase is a prokaryotic kinase or a fungal kinase.

In one embodiment the microbial kinase is a serine/threonine kinase.

In one embodiment the microbial kinase is a bacterial kinase.

In one embodiment the microbial kinase is a yeast kinase.

In one embodiment the microbial kinase is of the Hanks family.

In one embodiment the kinase is selected from the group consisting of YabT (SEQ ID NO: 5 or SEQ ID NO: 6), PrkD (SEQ ID NO: 7), PrkC (SEQ ID NO: 8) and PKNB1 (SEQ ID NO: 9) or functional variants thereof having at least 60% sequence identity, similarity or homology thereto, such as at least 70%, such as at least 80%, such as at least 85%, such as at least 90%, such as at least 95%, such as at least 98% sequence identity to any one of YabT (SEQ ID NO: 5), PrkD (SEQ ID NO: 7), PrkC (SEQ ID NO: 8) and PKNB1 (SEQ ID NO: 9).

In one embodiment the microbial kinase is YabT as set forth in SEQ ID NO: 5, or a functional variant thereof having at least 60% sequence identity, similarity or homology thereto, such as at least 70%, such as at least 80%, such as at least 85%, such as at least 90%, such as at least 95%, such as at least 98% sequence identity.

In one embodiment the microbial kinase is YabT as set forth in SEQ ID NO: 6, or a functional variant thereof having at least 60% sequence identity, similarity or homology thereto, such as at least 70%, such as at least 80%, such as at least 85%, such as at least 90%, such as at least 95%, such as at least 98% sequence identity.

In one embodiment the microbial kinase is PrkD as set forth in SEQ ID NO: 7, or a functional variant thereof having at least 70% sequence identity, similarity or homology thereto, such as at least 70%, such as at least 80%, such as at least 85%, such as at least 90%, such as at least 95%, such as at least 98% sequence identity.

In one embodiment the microbial kinase is PrkC as set forth in SEQ ID NO: 8, or a functional variant thereof having at least 60% sequence identity, similarity or homology thereto, such as at least 70%, such as at least 80%, such as at least 85%, such as at least 90%, such as at least 95%, such as at least 98% sequence identity.

In one embodiment the microbial kinase is PKNB1 as set forth in SEQ ID NO: 9, or a functional variant thereof having at least 60% sequence identity, similarity or homology thereto, such as at least 70%, such as at least 80%, such as at least 85%, such as at least 90%, such as at least 95%, such as at least 98% sequence identity.

### In Vitro Phosphorylation

In one embodiment the recombinant casein is phosphorylated outside the microbial host cell by a microbial kinase that is obtained separately and put in contact with the recombinant casein.

In one embodiment the recombinant casein is phosphorylated after the casein has been secreted.

In one embodiment the recombinant casein is phosphorylated in vitro.

### In Vivo Phosphorylation

In one embodiment the recombinant casein is phosphorylated inside the microbial host cell. This may be possible if the same microbial host cell has been engineered to recombinantly produce a casein according to the present disclosure and a microbial kinase according to the present disclosure.

In one embodiment the recombinant casein is phosphorylated in vivo.

### Functional Properties

For recombinant caseins to become a valuable substitute for casein obtained from mammalian milk, it is important that the recombinant casein holds at least some of the functional properties which casein obtained from mammalian milk holds. One of the most important properties is the ability to form micelles. The ability to form micelles is dependent on the ability of the casein to bind calcium, and the phosphorylation or presence of phosphomimetic residues in a casein is crucial for said casein to be able to bind calcium ions.

In one embodiment, the phosphorylated recombinant casein of the present disclosure binds calcium ions to comparable levels as casein obtained from mammalian milk, or from bovine milk.

In one embodiment, the phosphomimetic recombinant casein of the present disclosure binds calcium ions to comparable levels as casein obtained from mammalian milk or from bovine milk.

In one embodiment, the phosphorylated, phosphomimetic recombinant casein of the present disclosure binds calcium ions to comparable levels as casein obtained from mammalian milk or from bovine milk.

Other functional properties include for example coagulation, size of micelles, stability of micelles, gelling properties and emulsification properties. These parameters are critical for developing identical dairy products with functionality and structural properties similar to bovine caseins.

In one embodiment, the phosphorylated recombinant casein of the present disclosure, the phosphomimetic recombinant casein of the present disclosure and/or the phosphorylated, phosphomimetic recombinant casein of the present disclosure allows coagulation, gelation, emulsification and/or formation of stable micelles comparably to casein obtained from mammalian milk or from bovine milk.

### Micelle

The method of the current disclosure invention enables production of recombinant caseins, which are phosphorylated and/or phosphomimetic caseins, capable of forming micelles.

In one embodiment the present disclosure relates to a micelle comprising the recombinant phosphorylated and/or phosphomimetic casein produced as defined elsewhere in the description.

### Use of Recombinant Casein

The methods of the current disclosure provide recombinant casein, which is an alternative to casein obtained from mammalian milk. The recombinant casein can be used as an alternative to milk proteins in products which typically are derived from milk, that is dairy products, such as cheese and yoghurt. Moreover the recombinant casein of the current invention will provide useful as food additives.

In one embodiment the present disclosure relates to the use of a recombinant casein produced as defined elsewhere in the description, as a food ingredient/for preparing cheese, yogurt, or other liquid, solid or semi-solid milk products.

In one embodiment the present disclosure relates to a food product (including drinks) or a functional food product or a dairy product comprising recombinant casein produced as defined elsewhere in the description.

### Items

1. A system for producing one or more phosphorylated recombinant caseins, said system comprising:
   i. a microbial host cell expressing:
      a. at least one recombinant casein selected from the group consisting of alpha-casein S1 (αS1), alpha-casein S2 (αS2), beta-casein and kappa-casein and
      b. at least one microbial kinase;
   or
   ii. a microbial host cell expressing at least one casein selected from the group consisting of alpha-casein S1 (αS1), alpha-casein S2 (αS2), beta-casein and kappa-casein, and
      a composition comprising at least one microbial kinase,
         whereby said system produces one or more phosphorylated recombinant casein.
2. A system for producing one or more phosphomimetic recombinant caseins, said system comprising a microbial host cell expressing:
   i. at least one recombinant casein selected from the group consisting of alpha-casein S1 (αS1), alpha-casein S2 (αS2), beta-casein and kappa-casein, wherein one or more amino acid residues that are phosphorylated in a native mammalian casein are substituted with one or more phosphomimetic amino acids; and
   ii. optionally at least one microbial kinase capable of phosphorylating said phosphomimetic casein at residues which are phosphorylated in a native mammalian casein, and which have not been substituted with phosphomimetic amino acids,
   whereby said system is capable of producing one or more phosphomimetic recombinant casein.
3. The system according to any one of the preceding items, wherein the recombinant casein is a recombinant variant of a native mammalian casein, optionally wherein said mammalian casein is that found in milk from a mammal selected from the group consisting of human, cow, buffalo, zebu, yak, equines, sheep, goat, horse, donkey, camel, deer, and reindeer.
4. The system according to any one of the preceding items, wherein a native mammalian casein is a casein produced in a mammal, such as a casein produced in human, cow, buffalo, zebu, yak, equines, sheep, goat, horse, donkey, camel, deer, and reindeer, and wherein said native mammalian casein is naturally phosphorylated at one or more native phosphorylation sites.
5. The system according to any one of the preceding items, wherein the kinase is capable of phosphorylating one or more amino acid residues of said recombinant casein at its native phosphorylation sites.
6. The system according to any one of the preceding items, wherein the recombinant casein is phosphorylated inside the microbial host cell.
7. The system according to any one of the preceding items, wherein the recombinant casein is phosphorylated outside the microbial host cell.
8. The system according to any one of the preceding items, wherein the recombinant casein is phosphorylated after the casein has been secreted.
9. The system according to any one of the preceding items, wherein the recombinant casein is phosphorylated at one or more native phosphorylation sites, such as phosphorylated at least at two native phosphorylation sites, such as phosphorylated at least at three native phosphorylation sites, such as phosphorylated at least at four native phosphorylation sites, such as phosphorylated at least at five native phosphorylation sites, such as phosphorylated at least at six native phosphorylation sites, such as phosphorylated at least at seven native phosphorylation sites, such as phosphorylated at least at eight native phosphorylation sites, such as phosphorylated at least at nine native phosphorylation sites, such as phosphorylated at ten native phosphorylation sites, such as phosphorylated at eleven native phosphorylation sites, such as phosphorylated at twelve native phosphorylation sites, such as phosphorylated at thirteen native phosphorylation sites, such as phosphorylated at fourteen native phosphorylation sites, such as phosphorylated at fifteen native phosphorylation sites.
10. The system according to any one of the preceding items, wherein the recombinant casein is phosphorylated at all its native phosphorylation sites.
11. The casein according to any one of the preceding items, wherein the recombinant casein has a degree of phosphorylation at each native phosphorylation site of at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as about 100%.
12. The system according to any one of the preceding items, wherein the microbial host cell is a bacterial cell, a fungal cell or a yeast cell.
13. The system according to any one of the preceding items, wherein the microbial host cell is a bacterial cell, which belongs to a genus selected from the group consisting of lactic acid bacteria, for example Lactococcus spp. and Lactobacillus spp., Escherichia spp., Corynebacterium spp., Pseudomonas spp., Streptomyces spp. and Bacillus spp.
14. The system according to any one of the preceding items, wherein the microbial host cell is a bacterial host cell, which belongs to a species selected from the group consisting of species selected from Bacillus subtilis and Escherichia coli.
15. The system according to any one of the preceding items, wherein the microbial host cell is a fungal host cell, which belongs to a genus selected from the group consisting of Kluyveromyces spp., Pichia spp., Saccharomyces spp., Tetrahymena spp., Yarrowia spp., Hansenula spp., Blastobotrys spp., Aspergillus spp., Candida spp., Zygosaccharomyces spp., Trichoderma spp., Chrysosporium spp., Fusarium spp., and Neurospora spp. and Debaryomyces spp.
16. The system according to any one of the preceding items, wherein the microbial host cell is a fungal host cell, which belongs to a species selected from the group consisting of Trichoderma reesei, Aspergillus vadensis, Aspergillus oryzae and Aspergillus niger.
17. The system according to any one of the preceding items, wherein the microbial kinase is a prokaryotic kinase or a fungal kinase.
18. The system according to any one of the preceding items, wherein the microbial kinase is a serine/threonine kinase.
19. The system according to any one of the preceding items, wherein the microbial kinase is a bacterial kinase.
20. The system according to any one of the preceding items, wherein the microbial kinase is a yeast kinase.
21. The system according to any one of the preceding items, wherein the microbial kinase is of the Hanks family.
22. The system according to any one of the preceding items, wherein the kinase is selected from the group consisting of YabT (SEQ ID NO:5 or SEQ ID NO: 6), PrkD (SEQ ID NO: 7), PrkC (SEQ ID NO: 8) and PKNB1 (SEQ ID NO: 9) or functional variants thereof having at least 60% sequence identity, similarity or homology thereto, such as at least 70%, such as at least 80%, such as at least 85%, such as at least 90%, such as at least 95%, such as at least 98% sequence identity.
23. The system according to any one of the preceding items, wherein the microbial kinase is YabT as set forth in SEQ ID NO: 5, or a functional variant thereof having at least 70% sequence identity, similarity or homology thereto.
24. The system according to any one of the preceding items, wherein the microbial kinase is PrkD as set forth in SEQ ID NO: 7, or a functional variant thereof having at least 70% sequence identity, similarity or homology thereto.
25. The system according to any one of the preceding items, wherein the recombinant casein is selected from the group consisting of SEQ ID NO: 1, SEQ ID NO: 3, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a variant thereof having at least 90% sequence identity, similarity or homology thereto.
26. The system according to any one of the preceding items, wherein the recombinant casein is recombinant bovine casein.
27. The system according to item 26, wherein the recombinant bovine casein is selected from the group consisting of alpha-casein S1 (αS1), alpha-casein S2 (αS2), beta-casein and kappa-casein.
28. The system according to any one of items 26 to 27, wherein the recombinant bovine alpha-S1 casein (αS1) as set forth in SEQ ID NO: 1, is phosphorylated at least at one of the following positions: S46, S48, S64, S66, S67, S68, and S75.
29. The system according to any one of items 26 to 27, wherein the recombinant bovine alpha-S1 casein (αS1) as set forth in SEQ ID NO: 14, is phosphorylated at least at one of the following positions: S56, S61, S63, S79, S81, S82, S83, or S90.
30. The system according to any one of items 26 to 27, wherein the recombinant bovine alpha-S2 casein (αS2) as set forth in SEQ ID NO: 20, is phosphorylated at least at one of the following positions: S23, S24, S25, S28, S46, S71, S72, S73, S76, S144, S146, S150, or S158.
31. The system according to any one of items 26 to 27, wherein the recombinant bovine beta casein as set forth in SEQ ID NO: 3, is phosphorylated at least at one of the following positions: S15, S17, S18, S19 and S35.
32. The system according to any one of items 26 to 27, wherein the recombinant bovine beta casein as set forth in SEQ ID NO: 26, is phosphorylated at least at one of the following positions: S30, S32, S33, S34 and S50.
33. The system according to any one of items 26 to 27, wherein the recombinant bovine kappa casein as set forth in SEQ ID NO: 32, is phosphorylated at least at one of the following positions: S148, T166, S170, or S187.
34. The system according to any one of the preceding items wherein the recombinant casein is selected from the group consisting of human alpha-casein S1 (αS1), human alpha-casein (αS2), human beta-casein and human kappa-casein.
35. The system according to any one of the preceding items wherein the recombinant human alpha-S1 casein as set forth in SEQ ID NO: 10 is phosphorylated at least at one of the following positions: S31, S33, S41, S71, S85, S86, S88, S89, S90, or S91.
36. The system according to any one of the preceding items wherein the recombinant human beta casein as set forth in SEQ ID NO: 22, is phosphorylated at least at one of the following positions: T18, S21, S23, S24, or S25.
37. The system according to any one of the preceding items wherein the recombinant human kappa casein as set forth in SEQ ID NO: 28 is phosphorylated at positions T157.
38. The system according to any one of the preceding items, wherein said host cell comprises at least one protease deficiency rendering said host cell completely or partially deficient in the production and/or function of one or more proteases.
39. The system according to any one of the preceding items, wherein said host cell expresses at least one chaperone protein.
40. The system according to any one of the preceding items, wherein the recombinant casein is phosphorylated in vivo.
41. The system according to any one of the preceding items, wherein the recombinant casein is phosphorylated in vitro.
42. The system according to any one of the preceding item, wherein one or more amino acids have been substituted with a phosphomimetic amino acid.
43. The system according to any one of the preceding items, wherein one or more serine and/or threonine residues have been substituted with a phosphomimetic amino acid.
44. The system according to any one of the preceding items wherein one or more amino acid residues which are phosphorylated in the native mammalian casein have been substituted with one or more phosphomimetic amino acids.
45. The system according to any one of the preceding items, wherein one or more serine and/or threonine residues of the recombinant casein, such as of including alpha casein (αS1) as set forth in any one of SEQ ID NOs: 1, 10, 11, 12, 13 14 and 15, alpha casein (αS2) as set forth in any one of SEQ ID NOs: 16, 17, 18, 19, 20 and 21, beta casein as set forth in any one of SEQ ID NOs: 3, 22, 23, 24, 25, 26, and 27, and kappa casein as set forth in any one of SEQ ID NO 28, 29, 30, 31, 32 and 33, have been substituted with one or more phosphomimetic amino acids.
46. The system according to any one of the preceding items, wherein the recombinant casein is bovine alpha casein (αS1) as set forth in SEQ ID NO: 1, comprising a phosphomimetic amino acid at least at one of the following positions: S46, S48, S64, S66, S67, S68, and S75.
47. The system according to any one of the preceding items, wherein the recombinant casein is bovine alpha-S1 casein as set forth in SEQ ID NO: 14, comprising a phosphomimetic amino acid at least at one of the following positions: S56, S61, S63, S79, S81, S82, S83, or S90.
48. The system according to any one of the preceding items, wherein the recombinant casein is bovine alpha-S2 casein as set forth in SEQ ID NO: 20, comprising a phosphomimetic amino acid at least at one of the following positions: S23, S24, S25, S28, S46, S71, S72, S73, S76, S144, S146, S150, or S158.
49. The system according to any one of the preceding items, wherein the recombinant casein is bovine beta casein as set forth in SEQ ID NO: 3, comprising a phosphomimetic amino acid at least at one of the following positions: S15, S17, S18, S19 and S35.
50. The system according to any one of the preceding items, wherein the recombinant casein is bovine beta casein as set forth in SEQ ID NO: 26, comprising a phosphomimetic amino acid at least at one of the following positions: S30, S32, S33, S34 and S50.
51. The system according to any one of the preceding items, wherein the recombinant casein is bovine kappa casein as set forth in SEQ ID NO: 32, comprising a phosphomimetic amino acid at least at one of the following positions: S148, T166, S170, or S187.
52. The system according to any one of the preceding items, wherein the recombinant casein is human alpha-S1 casein as set forth in SEQ ID NO: 10, comprising a phosphomimetic amino acid at least at one of the following positions: S31, S33, S41, S71, S85, S86, S88, S89, S90, or S91.
53. The system according to any one of the preceding items, wherein the recombinant casein is human beta casein as set forth in SEQ ID NO: 22, comprising a phosphomimetic amino acid at least at one of the following positions: T18, S21, S23, S24, or S25.
54. The system according to any one of the preceding items, wherein the recombinant casein is human kappa casein as set forth in SEQ ID NO: 28, comprising a phosphomimetic amino acid at position T157.
55. The system according to any one of the preceding items 2 to 45, wherein the phosphomimetic amino acid is an unnatural amino acid.
56. The system according to any one of items 2 to 55, wherein the phosphomimetic amino acid carries negatively charged functional groups.
57. The system according to any one of items 2 to 56, wherein the phosphomimetic amino acid carries carboxylic acid groups.
58. The system according to any one of 2 to 57, wherein each phosphomimetic amino acid is independently selected from the group consisting of aspartic acid, glutamic acid and/or combinations thereof.
59. A phosphorylated recombinant casein obtained using a system according to any one of items 1 to 58.

Phosphomimetic casein represented by the following sequences: SEQ ID NO: 1, SEQ ID NO: 3, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, or a variant thereof, wherein said variant has at least 70% sequence identity to any one of SEQ ID NO: 1, SEQ ID NO: 3, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, and wherein said variant comprises at least one or more phosphomimetic amino acids at one or more of positions corresponding to positions S46, S48, S64, S66, S67, S68, and S75 of SEQ ID NO: 1; or positions of S15, S17, S18, S19 and S35 of SEQ ID NO: 3; or positions S31, S33, S41, S71, S85, S86, S88, S89, S90, S91 of SEQ ID NO: 10; or positions S33, S83, S85, S86, S87, S88 of SEQ ID NO: 11; or positions S27, S56, S61, S63, S79, S80, S81, S82, S83, S90, S130 of SEQ ID NO: 12; or positions S61, S63, S79, S80, S81, S82, S83, S90, S130 of SEQ ID NO: 13; or positions S56, S61, S63, S79, S81, S82, S83, S90 of SEQ ID NO: 14; or positions S63, S79, S80, S81, S82, S83, S90 of SEQ ID NO: 15; or positions S23, S24, S25, S28, S47, S68, S123, S125, S128, S136 of SEQ ID NO: 17; or positions S23, S24, S25, S72, S73, S74, S77, S145, S147, S151, S159 of SEQ ID NO: 18; or positions S23, S24, S25, S72, S73, S74, S77, S145, S147, S151, S159 of SEQ ID NO: 19; or positions S23, S24, S25, S28, S46, S71, S72, S73, S76, S144, S146, S150, S158 of SEQ ID NO:20; or positions T18, S21, S23, S24, S25 of SEQ ID NO:22; or positions T27, S30, S32, S33, S34 of SEQ ID NO:24; or positions T27, S30, S32, S33, S34 of SEQ ID NO:25; or positions S30, S32, S33, S34, S50 of SEQ ID NO:26; or positions S30, S32, S33, S34 of SEQ ID NO:27; or position T157 of SEQ ID NO: 28; or positions of S148, S172, S189 SEQ ID NO:30; or positions S148, T166, S170, S187 of SEQ ID NO:31; or positions S148, T166, S170, S187 of SEQ ID NO: 32.
60. An expression system for expression of phosphorylated recombinant casein in a microbial host cell, comprising:
   i. a first nucleic acid encoding at least one recombinant casein selected from the group consisting of alpha-casein S1 (αS1), alpha-casein (αS2), beta-casein and kappa-casein; and
   ii. a second nucleic acid encoding a kinase.
61. The expression system according to item 60, wherein the at least one recombinant selected from the group consisting of SEQ ID NO: 1, SEQ ID NO: 3, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a variant thereof having at least 90% sequence identity, similarity, or homology thereto.
62. The expression system according to any one of items 60 to 61, wherein the kinase is YabT as set forth in SEQ ID NO: 5 or SEQ ID NO: 6, PrkD as set forth in SEQ ID NO: 7, PrkC as set forth in SEQ ID NO: 8, PKNB1 as set forth in SEQ ID NO: 9 or functional variants thereof having at least 70% sequence identity, similarity, or homology to the sequences.
63. The expression system according to any one of items 60 to 62, wherein the nucleic acid encoding a casein and the nucleic acid encoding a kinase are expressed in a microbial host cell.
64. The expression system according to any one of items 60 to 63, wherein the nucleic acid encoding at least one casein is expressed in a first microbial host cell, and wherein the nucleic acid encoding at least one kinase is expressed in a second microbial host cell.
65. A host cell comprising the system according to any one of items 1 to 64, wherein said host cell is capable of producing recombinant casein.
66. A method of producing recombinant casein in a microbial host cell, comprising the steps of:
   i. Providing a host cell according to item 65;
   ii. incubating said host cell in a medium, under conditions allowing growth of the host cell, whereby at least one recombinant casein is produced; and
   iii. incubating the at least one casein with at least one microbial kinase,
      whereby phosphorylated recombinant casein is produced.
67. A method of producing recombinant casein in a microbial host cell, comprising the steps of:
   i. Providing a host cell according to item 65; and
   ii. incubating said host cell in a medium, under conditions allowing growth of the host cell of at least one phosphomimetic casein,
      whereby at least one phosphomimetic recombinant casein is produced.
68. A method of producing recombinant casein in a microbial host cell, comprising the steps of:
   i. Providing a host cell according to item 65; and
   ii. incubating said host cell in a medium, under conditions sufficient to allow for production of at least one phosphomimetic casein, whereby at least one phosphomimetic casein is produced;
   iii. incubating the at least one casein with at least one microbial kinase,
      whereby phosphorylated and/or phosphomimetic recombinant casein is produced.
69. The method according to any one of items 66 or 68, further comprising a step of recovering the recombinant casein.
70. Use of the system according to any one of the items 1 to 60 for production of at least one recombinant casein of interest.
71. Use of the expression system according to any one of the items 60 to 64, for production of at least one recombinant casein of interest.
72. Use of the methods according to any one of the items 66 to 68 for production of at least one recombinant casein of interest.
73. A food product (including drinks) or a functional food product or a dairy product comprising recombinant casein produced according to any one of the items 1 to 69.
74. Use of the recombinant casein produced according to any one of the items 1 to 69 as a food ingredient/for preparing yoghurt/cheese.
75. A micelle comprising the recombinant casein produced according to any one of the items 1 to 69.

### EXAMPLES

### Example 1. In Vitro Phosphorylation of Caseins Phosphorylation by Bovine and Human Kinase Fam20C

### Aim

Golgi-enriched fraction casein kinases with the consensus motif S-X-E/ps from the lactating mammary gland has been suggested in the prior art to be responsible for phosphorylating caseins (Moore et al, 1985). Fam20C from the GEF-CK was identified to be the one that specifically phosphorylates at S-X-E motifs (Tagliabracci et al, 2012).

The aim of the experiment was to express Fam20C from both bovine and human origin to test for phosphorylation of the caseins both in vitro and in vivo.

### Materials and Methods

Expression vectors pET15_BF20C and pET15_HF20C were generated by introducing the gene coding for bovine and human Fam20C respectively under the control of T7 promoter. Expression vector pREP4 GroESL was generated by introducing the gene coding for GroES/L under the control of T5 promoter. The expression vector containing the kinase (pET15_BF20C/pET15_HF20C) and the chaperone (pREP4 GroESL) was introduced into the host E. coli BL21 (DE3).

A colony was cultured overnight in LB broth with respective antibiotics of the vectors at 37 degrees with 200 rpm. A subculture was prepared after 16-18 hours and grown for 2-3 hours until the OD₆₀₀ reached between 0.6-0.8. Then 0.4 mM of IPTG (final concentration) was added and the culture was grown for 6 hours at 30 degrees to induce expression of the proteins. The cells were harvested, lysed using a sonicator and analyzed on SDS PAGE for identifying the target proteins from the soluble fraction. For recovering the insoluble proteins, 8M urea was used. The insoluble fraction was also analyzed on SDS-PAGE.

### Results

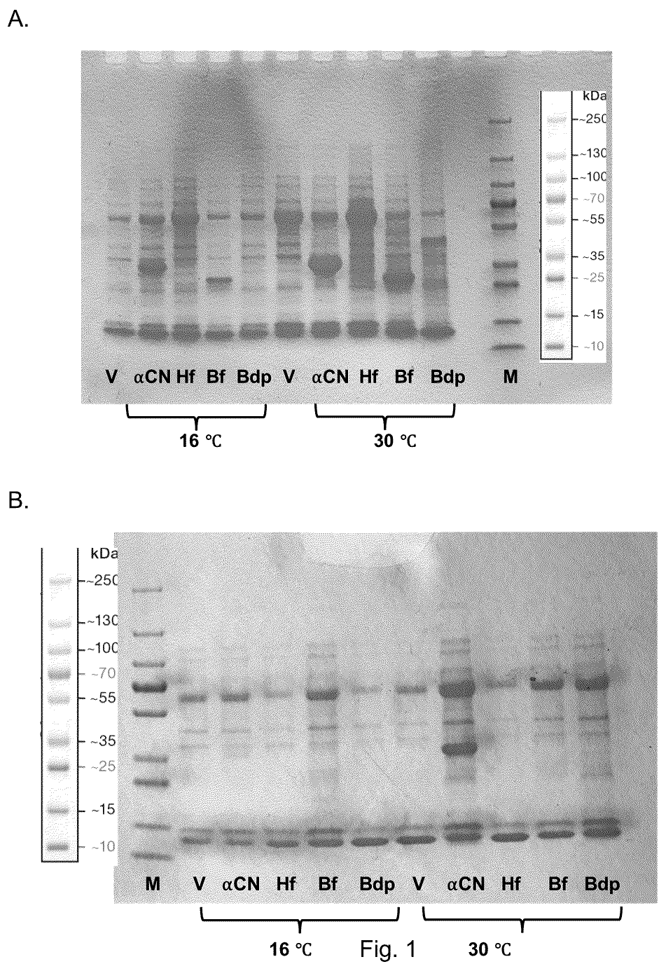

The kinases were produced as aggregates as shown in FIG. 1 and there was no visible expression of proteins from the soluble fraction of the cell lysate.

### Conclusions

The expression of bovine and human kinase Fam20C in soluble form was difficult to achieve. These proteins when expressed in E. coli formed inclusion bodies, and therefore we were unable to test activity and specificity (FIG. 1).

### Example 2. In Vitro Phosphorylation of Caseins-Phosphorylation of α-Casein by Microbial Kinases

### Aim

To investigate the in vitro phosphorylation of α-casein by PKNB-CGL0041-BAB97434 from C. glutamicum; PrkC, YabT, and PrkD from B. subtilis. The specific objectives were to identify the phosphorylation sites on αₛ₁-casein catalyzed by these kinases, compare the phosphorylation patterns with native bovine phosphorylation sites, and assess the efficiency and specificity of each kinase in phosphorylating α-casein.

### Materials and Methods

Bovine αₛ₁-casein (SEQ ID NO: 1) and the kinases were expressed individually and purified from E. coli BL21 (DE3). Casein was constructed using the pET15b vector and kinase using pQE30 vector. The strain with kinase plasmid is also transformed with pREP4 GroESL vector with the chaperones. The proteins were expressed in separate strains. αₛ₁-casein phosphorylation reactions were carried out using 3 UM PKNB-CGL0041-BAB97434 from C. glutamicum; PrkC, YabT, and PrkD from B. subtilis, along with 15 μM αₛ₁-casein. The reaction mixture, totaling 35 μl, consisted of the following components: 10 mM ATP, 5 mM MgCl₂, and 50 mM Tris-HCl at pH 7.4. Following a 2-hour incubation at 37° C., the sample was stored at −80° C. and sent for mass spectrometry (MS) analysis.

### Results

In vitro phosphorylation MS results, obtained by following the protocol of Beausoleil et al, 2006, demonstrated that PKNB and PrkC phosphorylated 6 sites in αₛ₁-casein each, of which only one and two sites, respectively, were similar to native bovine phosphorylation sites (showed with * in the table below). On the other hand, PrkD and YabT phosphorylated 13 and 10 sites, respectively, with 9 and 8 sites showing similarity to native phosphorylation sites.

| | Microbial Kinases | | | |
|---|---|---|---|---|
| Alpha casein | PKNB1 | PrkC | PrkD | YabT |
| Phosphorylation | (SEQ ID | (SEQ ID | (SEQ ID | (SEQ ID |
| site | NO: 9) | NO: 8) | NO: 7) | NO: 5) |
| *S56 | | + | ++ | +++ |
| *S61 | | | + | ++ |
| *S63 | | | + | ++ |
| *S79 | | | + | +++ |
| *S81 | | | + | +++ |
| *S82 | | | + | +++ |
| *S83 | | | + | +++ |
| *S90 | | | + | ++ |
| S103 | + | + | + | + |
| Y109 | + | + | | |
| *S130 | + | + | +++ | |
| S137 | + | + | + | + |
| Y159 | + | + | + | |
| Y161 | | | + | |
| Y188 | + | | | |

### Conclusions

PrkD exhibited the capability to phosphorylate all 9 native phosphorylation sites, while YabT could phosphorylate 8 out of the 9 native phosphorylation sites with a higher intensity.

### Example 3. In Vitro Phosphorylation of Caseins-Phosphorylation of β-Casein by Microbial Kinases

### Aim

To investigate the in vitro phosphorylation of β-casein by PKNB-CGL0041-BAB97434 from C. glutamicum; PrkC, YabT, and PrkD from B. subtilis. The specific objectives were to identify the phosphorylation sites on β-casein catalyzed by these kinases, compare the phosphorylation patterns with native bovine phosphorylation sites, and assess the efficiency and specificity of each kinase in phosphorylating β-casein.

### Materials and Methods

Bovine β-casein (SEQ ID NO: 3) and the kinases were expressed individually and purified from E. coli BL21 (DE3). Casein was constructed using the pET15b vector and kinase using pQE30 vector. The strain with kinase plasmid is also transformed with pREP4 GroESL vector with the chaperones. The proteins were expressed in separate strains. β-casein phosphorylation reactions were carried out using 3 μM PKNB-CGL0041-BAB97434 from C. glutamicum; PrkC, YabT, and PrkD from B. subtilis, along with 15 μM β-casein. The reaction mixture, in total 35 μl, consisted of the following components: 10 mM ATP, 5 mM MgCl2, and 50 mM Tris-HCl at pH 7.4. Following a 2-hour incubation at 37° C., the sample was stored at −80° C. and sent for Mass spectrometry analysis.

### Results

In vitro phosphorylation MS results, obtained by following the protocol of Beausoleil et al, 2006, demonstrated that PKNB and PrkC phosphorylated 2 sites in β-casein each, of which only one site were similar to native bovine phosphorylation sites (showed with * in the table below). On the other hand, PrkD and YabT phosphorylated 5 sites each, with 3 sites showing similarity to native phosphorylation sites.

| | Microbial kinases | | | |
|---|---|---|---|---|
| Beta casein | PKNB1 | PrkC | PrkD | YabT |
| Phosphorylation | (SEQ ID | (SEQ ID | (SEQ ID | (SEQ ID |
| sites | NO: 9) | NO: 8) | NO: 7) | NO: 5) |
| *S30 | + | + | + | ++ |
| *S32 | | | + | ++ |
| T39 | +++ | ++ | + | ++ |
| *S50 | | | + | ++ |
| T56 | | | | + |
| S337 | | | + | |

### Conclusions

PrkD and YabT exhibited the capability to phosphorylate 3 native phosphorylation sites in β-casein, while YabT showed higher intensity.

### Example 4. Co-expression of kinase (B. subtilis PrkD) and casein

### Aim

To facilitate phosphorylation of caseins in vivo through co-expression of caseins and PrkD in E. coli BL21 (DE3).

### Materials and Methods

The expression vector pET αₛ₁_PrkD/pETβ_PrkD was generated by inserting the casein (bovine αₛ₁-casein (SEQ ID NO: 1) or bovine β-casein (SEQ ID NO: 3)) and PrkD (SEQ ID NO: 7) under the control of T7 promoter. The expression vector containing the casein (pET αₛ₁_PrkD/pET β_PrkD) and the chaperone (pREP4 GroESL) was introduced into the host E. coli BL21 (DE3).

A colony was cultured overnight in LB broth with respective antibiotics of the vectors at 37 degrees with 200 rpm. A subculture was prepared after 16-18 hours and grown for 2-3 hours until the OD₆₀₀ reached between 0.6-0.8. Then 0.4 mM of IPTG (final concentration) was added and the culture was grown for 6 hours at 30 degrees to induce expression of the proteins. The cells were harvested, lysed using a sonicator and analyzed on SDS PAGE for identifying the target proteins from the soluble fraction.

### Results

Both casein and PrkD were expressed in soluble form. The expression levels of PrkD was low, however, for phosphorylation even low amounts of kinase is sufficient.

### Conclusions

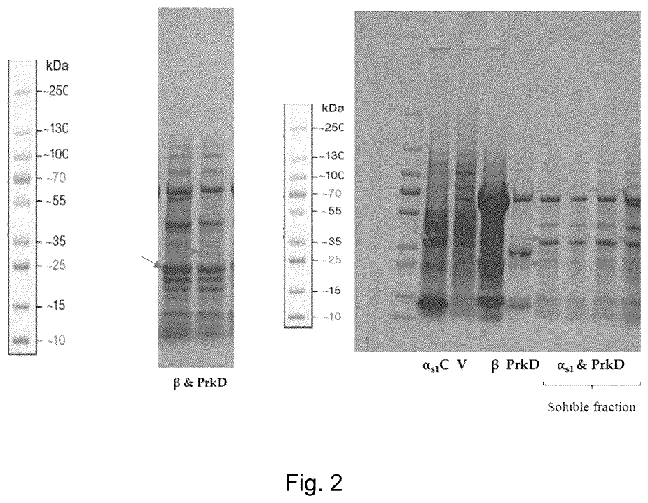

B. subtilis PrkD and caseins α_{S1} and β were successfully co-expressed (FIG. 2).

### Example 5. Co-Expression of Kinase (B. subtilis YabT) and Casein

### Aim

To facilitate in vivo phosphorylation of αₛ₁-casein through co-expression of caseins and YabT in E. coli BL21 (DE3).

### Materials and Methods

The expression vector pET αₛ₁_YabT was generated by inserting the casein and YabT under the control of T7 promoter. The expression vector containing the casein (bovine αₛ₁-casein (SEQ ID NO: 1) or bovine β-casein (SEQ ID NO: 3)) and YabT (SEQ ID NO: 6)), (expression vector pET αₛ₁_YabT) and the chaperone (pREP4 GroESL) was introduced into the host E. coli BL21 (DE3).

A colony was cultured overnight in LB broth with respective antibiotics of the vectors at 37 degrees with 200 rpm. A subculture was prepared after 16-18 hours and grown for 2-3 hours until the OD₆₀₀ reached between 0.6-0.8. Then 0.4 mM of IPTG (final concentration) was added and the culture was grown for 6 hours at 30 degrees to induce expression of the proteins. The cells were harvested, lysed using a sonicator and analyzed on SDS PAGE for identifying the target proteins from the soluble fraction.

### Results

Both α_{S1} casein and YabT were expressed in soluble form.

### Conclusions

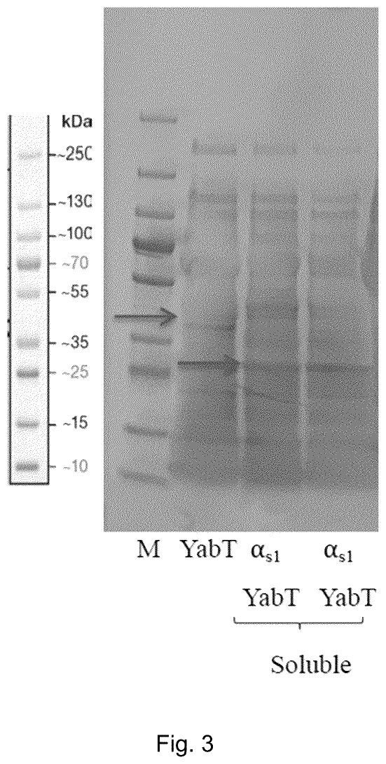

B. subtilis YabT and casein α_{S1} were successfully co-expressed (FIG. 3).

### Example 6. Phosphomimetic Modification

### Aim

To express phosphomimetic variants of caseins in E. coli BL21 (DE3) as an alternative to phosphorylation.

### Materials and Methods

The expression vector pET αₛ₁_Asp/pETβ_Asp was generated by inserting the casein phosphomimetic variants (Δₛ₁-casein (SEQ ID NO: 2) or bovine β-casein (SEQ ID NO: 4)) under the control of T7 promoter. The caseins were synthesized by replacing the potential serine sites into aspartic acid. The expression vector containing the casein (pET αₛ₁_Asp/pET β_Asp) and the chaperone (pREP4 GroESL) was introduced into the host E. coli BL21 (DE3).

A colony was cultured overnight in LB broth with respective antibiotics of the vectors at 37 degrees with 200 rpm. A subculture was prepared after 16-18 hours and grown for 2-3 hours until the OD₆₀₀ reached between 0.6-0.8. Then 0.4 mM of IPTG (final concentration) was added and the culture was grown for 6 hours at 30 degrees to induce expression of the proteins. The cells were harvested, lysed using a sonicator and analyzed on SDS PAGE for identifying the target proteins from the soluble fraction.

### Results

The phosphomimetic variants of the caseins were successfully expressed in soluble form.

### Conclusions

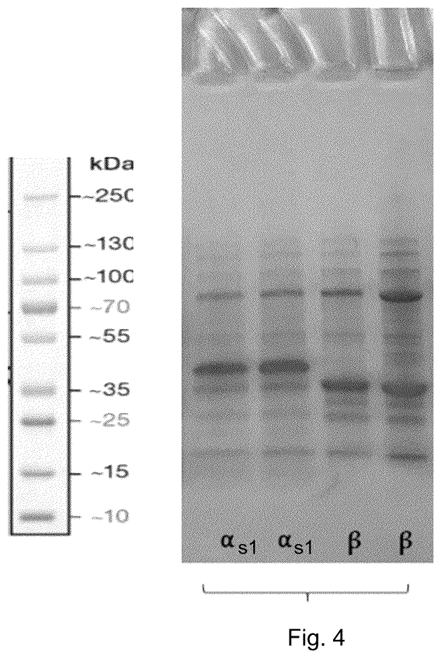

Phosphomimetic variants—αₛ₁ & β-casein having serine replaced with aspartic acid were successfully expressed (FIG. 4).

### Example 7. Calcium Binding Assay

### Aim

To test the calcium binding properties of unmodified and modified caseins.

### Materials and Methods

1 mg of bovine casein (αₛ₁-casein, β-casein phosphorylated in vitro by microbial kinases YabT or PrkD) was incubated with 2.29 μM of Cacl₂ in 20 mM of Tris buffer, pH 8 at 37° C. for 1 hour. Commercially available bovine αₛ₁-casein and β-casein were used as positive controls. Dephosphorylated commercially available bovine αₛ₁-casein and β-casein were used as negative controls. The samples were centrifuged at 15000 rpm for 30 minutes. The supernatant was filtered using 10 kDa centrifugal filters. The flow through contained buffer with unbound ca ions which was then measured using Ca calorimetry assay kit from Sigma Aldrich. Caseins from Sigma was dephosphorylated were used as negative controls, along with Tris buffer with Cacl₂.

### Results

The positive controls α and β caseins from Sigma Aldrich, and the phosphorylated samples has less calcium ions (unbound ca) in the flowthrough compared to dephosphorylated caseins.

### Conclusion

Commercial caseins and the phosphorylated samples bind calcium ions to comparable levels.

Sequence overview
Bovine Alpha-s1-casein
(SEQ ID NO: 1)
RPKHPIKHQGLPQEVLNENLLRFFVAPFPEVFGKEKVNELSKDIG
SESTEDQAMEDIKQMEAESISSSEEIVPNSVEQKHIQKEDVPSER
YLGYLEQLLRLKKYKVPQLEIVPNSAEERLHSMKEGIHAQQKEPM
IGVNQELAYFYPELFRQFYQLDAYPSGAWYYVPLGTQYTDAPSFS
DIPNPIGSENSEKTTMPLW
Alpha-s1-casein (Ser→Asp)
(SEQ ID NO: 2)
RPKHPIKHQGLPQEVLNENLLRFFVAPFPEVFGKEKVNELSKDIG
DEDTEDQAMEDIKQMEAEDIDDDEEIVPNDVEQKHIQKEDVPSER
YLGYLEQLLRLKKYKVPQLEIVPNDAEERLHSMKEGIHAQQKEPM
IGVNQELAYFYPELFRQFYQLDAYPSGAWYYVPLGTQYTDAPSFS
DIPNPIGSENSEKTTMPLW
Bovine Beta-casein
(SEQ ID NO: 3)
RELEELNVPGEIVESLSSSEESITRINKKIEKFQSEEQQQTEDEL
QDKIHPFAQTQSLVYPFPGPIPNSLPQNIPPLTQTPVVVPPFLQP
EVMGVSKVKEAMAPKHKEMPFPKYPVEPFTESQSLTLTDVENLHL
PLPLLQSWMHQPHQPLPPTVMFPPQSVLSLSQSKVLPVPQKAVPY
PQRDMPIQAFLLYQEPVLGPVRGPFPIIV
Beta-casein (Ser→Asp)
(SEQ ID NO: 4)
RELEELNVPGEIVEDLDDDEESITRINKKIEKFQDEEQQQTEDEL
QDKIHPFAQTQSLVYPFPGPIPNSLPQNIPPLTQTPVVVPPFLQP
EVMGVSKVKEAMAPKHKEMPFPKYPVEPFTESQSLTLTDVENLHL
PLPLLQSWMHQPHQPLPPTVMFPPQSVLSLSQSKVLPVPQKAVPY
PQRDMPIQAFLLYQEPVLGPVRGPFPIIV
YabT (GenBank: SPU13583.1)
(SEQ ID NO: 5)
MLNWTPKLQSLSTPFAKLSTAAITPTGPAIREATLQFEKYAQEGA
CWQMMNDALTSLACSLKPGTTIKGKWNGNTYTLRKQLGKGANGIV
YLAETSDGHVALKVSDDSLSITSEVNVLKSFSKAQSVTMGPSFFD
TDDAYIPSANTKVSFYAMEYIKGPLLLKYVSDKGAEWIPVLMIQL
LSSLSVLHQQGWIFGDLKPDNLIVTGPPARIRCIDVGGTTKEGRA
IKEYTEFYDRGYWGYGTRKAEPSYDLFAVAMIMINSVHKKEFKKT
NQPKEQLRSLIEGNPLLQKYKKALFSALNGDYQSADEMKKDMLDA
GQKAAQRKQPIKASPQPATRQRQQKPRQGKITKTRYTPKQKPAKS
GGLFETTLIVISVLALYFAYIIFFLI
Truncated YabT-used for co-expression
(SEQ ID NO: 6)
MMNDALTSLACSLKPGTTIKGKWNGNTYTLRKQLGKGANGIVYLA
ETSDGHVALKVSDDSLSITSEVNVLKSFSKAQSVTMGPSFFDTDD
AYIPSANTKVSFYAMEYIKGPLLLKYVSDKGAEWIPVLMIQLLSS
LSVLHQQGWIFGDLKPDNLIVTGPPARIRCIDVGGTTKEGRAIKE
YTEFYDRGYWGYGTRKAEPSYDLFAVAMIMINSVHKKEFKKTNQP
KEQLRSLIEGNPLLQKYKKALFSALNGDYQSADEMKKDMLDAGQK
AAQRKQPIKASPQPATRQRQQKPRQGKITKTRYTPKQKPAKSGGL
FETTLI
PrkD (Bacillus subtilis 168 | BSU_02030)
(SEQ ID NO: 7)
MALKLLKKLLFDRPLKNGVILNHQYKIEECLGMGGYGLVYLCTDI
LAQTPYVLKQLRPTKAKKEKEKVRFQQEIKLLKNIHHPQIPGFID
EFIIDGQAYYVMQFIEGENIEELLFFRKQPFTELMALQLISQLLE
IIEYLHDRLIFHSDIRTPNIIINDGRLCLIDFGLAKQLTPEEMEE
IKVRKQDDFFDLGETLLFLLYSQYKGKKKKNGTWLEELTLTKEVT
LLLKRLLGIEEEYQHTASIREDLNRAIQSVT
PrkC (Bacillus subtilis)
(SEQ ID NO: 8)
MLIGKRISGRYQILRVIGGGGMANVYLAEDIILDREVAIKILRFD
YANDNEFIRRFRREAQSASSLDHPNIVSIYDLGEEDDIYYIVMEY
VEGMTLKEYITANGPLHPKEALNIMEQIVSAIAHAHQNQIVHRDI
KPHNILIDHMGNIKVTDFGIATALSSTTITHTNSVLGSVHYLSPE
QARGGLATKKSDIYALGIVLFELLTGRIPFDGESAVSIALKHLQA
ETPSAKRWNPSVPQSVENIILKATAKDPFHRYETAEDMEADIKTA
FDADRLNEKRFTIQEDEEMTKAIPIIKDEELAKAAGEKEAEVTTA
QENKTKKNGKRKKWPWVL
PKNB1_Cgl2751 (Corynebacterium glutamicum)
(SEQ ID NO: 9)
MYEVLGVIAHGGMGWIYLANDRNVSGRIVVLKGMMAQSSVQDQGT
AEAEREFLADITHPGIVKAYNFIDDPRVPGGFIVMEYVNGPSLKD
RCKAQPDGVLRVDLAIGYILELLPAMDYLHQRGVVYNDLKPENVI
ATEDQVKLIDLGAVTGIGAFGYIYGTKGFQAPEVATHGPSISSDI
FTIGRTLAALTMPLPVEDGVLAPGIPSPKNSPLLRRHLSFYRLLQ
RATADDPQHRFRNVSELRTQLYGVLREILAVRDGKQYPPQHSLFS
PQRSTFGTKHLVF
Human Alpha-s1 casein: P47710 CASA1_HUMAN
(SEQ ID NO: 10)
MRLLILTCLVAVALARPKLPLRYPERLQNPSESSEPIPLESREEY
MNGMNRQRNILREKQTDEIKDTRNESTQNCVVAEPEKMESSISSS
SEEMSLSKCAEQFCRLNEYNQLQLQAAHAQEQIRRMNENSHVQVP
FQQLNQLAAYPYAVWYYPQIMQYVPFPPFSDISNPTAHENYEKNN
VMLQW
Camel Alpha-s1 casein: 097943 CASA1_CAMDR
(SEQ ID NO: 11)
MKLLILTCLVAVALARPKYPLRYPEVFQNEPDSIEEVLNKRKILE
LAVVSPIQFRQENIDELKDTRNEPTEDHIMEDTERKESGSSSSEE
VVSSTTEQKDILKEDMPSQRYLEELHRLNKYKLLQLEAIRDQKLI
PRVKLSSHPYLEQLYRINEDNHPQLGEPVKVVTQEQAYFHLEPFP
QFFQLGASPYVAWYYPPQVMQYIAHPSSYDTPEGIASEDGGKTDV
MPQWW
Sheep Alpha-s1 casein: P04653 CAS A1_SHEEP
(SEQ ID NO: 12)
MKLLILTCLVAVALARPKHPIKHQGLSSEVLNENLLRFVVAPFPE
VFRKENINELSKDIGSESIEDQAMEDAKQMKAGSSSSSEEIVPNS
AEQKYIQKEDVPSERYLGYLEQLLRLKKYNVPQLEIVPKSAEEQL
HSMKEGNPAHQKQPMIAVNQELAYFYPQLFRQFYQLDAYPSGAWY
YLPLGTQYTDAPSFSDIPNPIGSENSGKITMPLW
Goat Alpha-s1 casein: P18626 CAS A1_CAPHI
(SEQ ID NO: 13)
MKLLILTCLVAVALARPKHPINHRGLSPEVPNENLLRFVVAPFPE
VFRKENINELSKDIGSESTEDQAMEDAKQMKAGSSSSSEEIVPNS
AEQKYIQKEDVPSERYLGYLEQLLRLKKYNVPQLEIVPKSAEEQL
HSMKEGNPAHQKQPMIAVNQELAYFYPQLFRQFYQLDAYPSGAWY
YLPLGTQYTDAPSFSDIPNPIGSENSGKTTMPLW
Bovine Alpha-s1 casein: P02662 CAS A1_BOVIN
(including bovine signal peptide,
highlighted)
(SEQ ID NO: 14)
MKLLILTCLVAVALARPKHPIKHQGLPQEVLNENLLRFFVAPFPE
VFGKEKVNELSKDIGSESTEDQAMEDIKQMEAESISSSEEIVPNS
VEQKHIQKEDVPSERYLGYLEQLLRLKKYKVPQLEIVPNSAEERL
HSMKEGIHAQQKEPMIGVNQELAYFYPELFRQFYQLDAYPSGAWY
YVPLGTQYTDAPSFSDIPNPIGSENSEKTTMPLW
Buffalo Alpha-s1 casein: 062823 CAS A1_BUBBU
(SEQ ID NO: 15)
MKLLILTCLVAVALARPKQPIKHQGLPQGVLNENLLRFFVAPFPE
VFGKEKVNELSTDIGSESTEDQAMEDIKQMEAESISSSEEIVPIS
VEQKHIQKEDVPSERYLGYLEQLLRLKKYNVPQLEIVPNLAEEQL
HSMKEGIHAQQKEPMIGVNQELAYFYPQLFRQFYQLDAYPSGAWY
YVPLGTQYPDAPSFSDIPNPIGSENSGKTTMPLW
Human Alpha-s2 casein: Tr Q8IUJ1 CASA2_HUMAN
(SEQ ID NO: 16)
MKFFIFTCLLAVALAHHEIKHSSSSSE
Camel Alpha-s2 casein: Sp 097944 CASA2_CAMDR
(SEQ ID NO: 17)
MKFFIFTCLLAVVLAKHEMDQGSSSEESINVSQQKFKQVKKVAIH
PSKEDICSTFCEEAVRNIKEVESAEVPTENKISQFYQKWKFLQYL
QALHQGQIVMNPWDQGKTRAYPFIPTVNTEQLSISEESTEVPTEE
STEVFTKKTELTEEEKDHQKFLNKIYQYYQTFLWPEYLKTVYQYQ
KTMTPWNHIKRYF
Sheep Alpha-s2 casein: Sp P04654 CAS A2_SHEEP
(SEQ ID NO: 18)
MKFFIFTCLLAVALAKHKMEHVSSSEEPINISQEIYKQEKNMAIH
PRKEKLCTTSCEEVVRNADEEEYSIRSSSEESAEVAPEEVKITVD
DKHYQKALNEINQFYQKFPQYLQYLYQGPIVLNPWDQVKRNAGPF
TPTVNREQLSTSEENSKKTIDMESTEVFTKKTKLTEEEKNRLNFL
KKISQYYQKFAWPQYLKTVDQHQKAMKPWTQPKTNAIPYVRYL
Goat Alpha-s2 casein: Sp P33049 CAS A2_CAPHI
(SEQ ID NO: 19)
MKFFIFTCLLAVALAKHKMEHVSSSEEPINIFQEIYKQEKNMAIH
PRKEKLCTTSCEEVVRNANEEEYSIRSSSEESAEVAPEEIKITVD
DKHYQKALNEINQFYQKFPQYLQYPYQGPIVLNPWDQVKRNAGPF
TPTVNREQLSTSEENSKKTIDMESTEVFTKKTKLTEEEKNRLNFL
KKISQYYQKFAWPQYLKTVDQHQKAMKPWTQPKTNAIPYVRYL
Bovine Alpha-s2 casein: Sp P02663 CAS A2_BOVIN
(SEQ ID NO: 20)
MKFFIFTCLLAVALAKNTMEHVSSSEESIISQETYKQEKNMAINP
SKENLCSTFCKEVVRNANEEEYSIGSSSEESAEVATEEVKITVDD
KHYQKALNEINQFYQKFPQYLQYLYQGPIVLNPWDQVKRNAVPIT
PTLNREQLSTSEENSKKTVDMESTEVFTKKTKLTEEEKNRLNFLK
KISQRYQKFALPQYLKTVYQHQKAMKPWIQPKTKVIPYVRYL
Buffalo Alpha-s2 casein: Tr E9NZN2 CAS A2_BUBBU
(SEQ ID NO: 21)
MKFFIFTCLLAVALAKHTMEHVSSSEESIISQETYKQEKNMAIHP
SKENLCSTFCKEVIRNANEEEYSIGSSSEESAEVATEEVKITVDD
KHYQKALNEINQFYQKFPQYLQYLYQGPIVLNPWDQVKRNAVPIT
PTLNREQLSTSEENSKKTVDMESTEVFTKKTKLTEEDKNRLNFLK
KISQHYQKFAWPQYLKTVYQYQKAMKPWTQPKTNVIPYVRYL
Human Beta-casein: Sp P05814 CASB_human
(SEQ ID NO: 22)
MKVLILACLVALALARETIESLSSSEESITEYKQKVEKVKHEDQQ
QGEDEHQDKIYPSFQPQPLIYPFVEPIPYGFLPQNILPLAQPAVV
LPVPQPEIMEVPKAKDTVYTKGRVMPVLKSPTIPFFDPQIPKLTD
LENLHLPLPLLQPLMQQVPQPIPQTLALPPQPLWSVPQPKVLPIP
QQVVPYPQRAVPVQALLLNQELLLNPTHQIYPVTQPLAPVHNPIS
V
Camel Beta-casein: Tr M1E4K4 CASB_CAMDR
(SEQ ID NO: 23)
MKVLILACLVALALAREKEEFKTAGEALESISSSEESITHINKQK
IEKFKIEEQQQTEDEQQDKIYTFPQPQSLVYSHTEPIPYPILPQN
FLPPLQPAVMVPFLQPKVMDVPKTKETIIPKRKEMPLLQSPVVPF
TESQSLTLTDLENLHLPLPLLQSLMYQIPQPVPQTPMIPPQSLLS
LSQFKVLPVPQQMVPYPQRAMPVQAVLPFQEPVPDPVRGLHPVPQ
PLVPVIA
Sheep Beta-casein: Sp P11839 CASB_SHEEP
(SEQ ID NO: 24)
MKVLILACLVALALAREQEELNVVGETVESLSSSEESITHINKKI
EKFQSEEQQQTEDELQDKIHPFAQAQSLVYPFTGPIPNSLPQNIL
PLTQTPVVVPPFLQPEIMGVPKVKETMVPKHKEMPFPKYPVEPFT
ESQSLTLTDVEKLHLPLPLVQSWMHQPPQPLPPTVMFPPQSVLSL
SQPKVLPVPQKAVPQRDMPIQAFLLYQEPVLGPVRGPFPILV
Goat Beta-casein: Sp P33048 CASB_CAPHI
(SEQ ID NO: 25)
MKVLILACLVALAIAREQEELNVVGETVESLSSSEESITHINKKI
EKFQSEEQQQTEDELQDKIHPFAQAQSLVYPFTGPIPNSLPQNIL
PLTQTPVVVPPFLQPEIMGVPKVKETMVPKHKEMPFPKYPVEPFT
ESQSLTLTDVEKLHLPLPLVQSWMHQPPQPLSPTVMFPPQSVLSL
SQPKVLPVPQKAVPQRDMPIQAFLLYQEPVLGPVRGPFPILV
Bovine Beta-casein: Sp P02666 CASB_BOVIN
(including bovine signal Peptide, highlighted)
used for numbering of phosphorylation sites
(SEQ ID NO: 26)
MKVLILACLVALALARELEELNVPGEIVESLSSSEESITRINKKI
EKFQSEEQQQTEDELQDKIHPFAQTQSLVYPFPGPIPNSLPQNIP
PLTQTPVVVPPFLQPEVMGVSKVKEAMAPKHKEMPFPKYPVEPFT
ESQSLTLTDVENLHLPLPLLQSWMHQPHQPLPPTVMFPPQSVLSL
SQSKVLPVPQKAVPYPQRDMPIQAFLLYQEPVLGPVRGPFPIIV
Buffalo Beta-casein: Sp Q9TSIO CASB_BUBBU
(SEQ ID NO: 27)
MKVLILACLVALALARELEELNVPGEIVESLSSSEESITHINKKI
EKFQSEEQQQMEDELQDKIHPFAQTQSLVYPFPGPIPKSLPQNIP
PLTQTPVVVPPFLQPEIMGVSKVKEAMAPKHKEMPFPKYPVEPFT
ESQSLTLTDVENLHLPLPLLQSWMHQPPQPLPPTVMFPPQSVLSL
SQSKVLPVPQKAVPYPQRDMPIQAFLLYQEPVLGPVRGPFPIIV
Human Kappa-casein: Sp P07498 CASK_human
(SEQ ID NO: 28)
MKSFLLVVNALALTLPFLAVEVQNQKQPACHENDERPFYQKTAPY
VPMYYVPNSYPYYGTNLYQRRPAIAINNPYVPRTYYANPAVVRPH
AQIPQRQYLPNSHPPTVVRRPNLHPSFIAIPPKKIQDKIIIPTIN
TIATVEPTPAPATEPTVDSVVTPEAFSESIITSTPETTTVAVTPP
TA
Camel Kappa-casein: Sp P79139 CASK_CAMDR
(SEQ ID NO: 29)
MKSFFLVVTILALTLPFLGAEVQNQEQPTCFEKVERLLNEKTVKY
FPIQFVQSRYPSYGINYYQHRLAVPINNQFIPYPNYAKPVAIRLH
AQIPQCQALPNIDPPTVERRPRPRPSFIAIPPKKTQDKTVNPAIN
TVATVEPPVIPTAEPAVNTVVIAEASSEFITTSTPETTTVQITST
EI
Sheep Kappa-casein: Sp P02669 CASK_SHEEP
(SEQ ID NO: 30)
MMKSFFLVVTILALTLPFLGAQEQNQEQRICCEKDERFFDDKIAK
YIPIQYVLSRYPSYGLNYYQQRPVALINNQFLPYPYYAKPVAVRS
PAQTLQWQVLPNAVPAKSCQDQPTAMARHPHPHLSFMAIPPKKDQ
DKTEIPAINTIASAEPTVHSTPTTEAVVNAVDNPEASSESIASAP
ETNTAQVTSTEV
Goat Kappa-casein: Sp P02670 CASK_CAPHI
(SEQ ID NO: 31)
MMKSFFLVVTILALTLPFLGAQEQNQEQPICCEKDERFFDDKIAK
YIPIQYVLSRYPSYGLNYYQQRPVALINNQFLPYPYYAKPVAVRS
PAQTLQWQVLPNTVPAKSCQDQPTTLARHPHPHLSFMAIPPKKDQ
DKTEVPAINTIASAEPTVHSTPTTEAIVNTVDNPEASSESIASAS
ETNTAQVTSTEV
Bovine Kappa-casein: Sp P02668 CASK_BOVIN
(SEQ ID NO: 32)
MMKSFFLVVTILALTLPFLGAQEQNQEQPIRCEKDERFFSDKIAK
YIPIQYVLSRYPSYGLNYYQQKPVALINNQFLPYPYYAKPAAVRS
PAQILQWQVLSNTVPAKSCQAQPTTMARHPHPHLSFMAIPPKKNQ
DKTEIPTINTIASGEPTSTPTTEAVESTVATLEDSPEVIESPPEI
NTVQVTSTAV
Buffalo Kappa-casein: Tr K9ZU53 CASK_BUBBU
(SEQ ID NO: 33)
YGLNYYQQKPVALINNQFLPYPYYAKPAAVRSPAQILQWQVLPNT
VPAKSCQAQPTTMTRHPHPHLSFMAIPPKKNQDKTEIPTINTIVS
VEPTSTPTTEAIENTVATLEASSDVIESVPETNTAQVTSTVV

### REFERENCES

Beausoleil, S A et al.: A probability-based approach for high-throughput protein phosphorylation analysis and site localization, Nature Biotechnology 24, 1285-1292 (2006)
Jimenez-Flores R, Richardson T, and Bisson L. F. Expression of bovine.beta.-casein in Saccharomyces cerevisiae and characterization of the protein produced in vivo. Journal of Agricultural and Food Chemistry 1990 38 (4), 1134-1141.
Moore A, Boulton A P, Heid H W, Jarasch E D, Craig R K. Purification and tissue-specific expression of casein kinase from the lactating guinea-pig mammary gland. Eur J Biochem. 1985 Nov. 4; 152 (3): 729-37. doi: 10.1111/j. 1432-1033.1985.tb09254.x. PMID: 3863754.
Tagliabracci V S, Engel J L, Wen J, Wiley S E, Worby C A, Kinch L N, Xiao J, Grishin N V, Dixon J E. Secreted kinase phosphorylates extracellular proteins that regulate biomineralization. Science. 2012 Jun. 1; 336 (6085): 1150-3.
Thurmond J M, Hards R G, Seipelt C T, Leonard A E, Hansson L, Strömqvist M, Byström M, Enquist K, Xu B C, Kopchick J J, Mukerji P. Expression and characterization of phosphorylated recombinant human beta-casein in Escherichia coli. Protein Expr Purif. 1997 July; 10 (2): 202-8.
Tubb, C., and T. Seba. 2019. Rethinking food and agriculture 2020-2030: The second domestication of plants and animals, the disruption of the cow, and the collapse of industrial livestock farming.

1. A system for producing one or more phosphorylated recombinant caseins, said system comprising:
i. a microbial host cell expressing:
a. at least one recombinant casein selected from the group consisting of alpha-casein S1 (αS1), alpha-casein S2 (αS2), beta-casein and kappa-casein and
b. at least one microbial kinase;
or
ii. a microbial host cell expressing at least one casein selected from the group consisting of alpha-casein S1 (αS1), alpha-casein S2 (αS2), beta-casein and kappa-casein, and
a composition comprising at least one microbial kinase,
whereby said system produces one or more phosphorylated recombinant casein.

2. The system according to claim 1, wherein the recombinant casein is a recombinant variant of a native mammalian casein, optionally wherein said mammalian casein is that found in milk from a mammal selected from the group consisting of human, cow, buffalo, zebu, yak, equines, sheep, goat, horse, donkey, camel, deer, and reindeer, and wherein said native mammalian casein is naturally phosphorylated at one or more native phosphorylation sites.

3. The system according to claim 1, wherein the microbial kinase is capable of phosphorylating one or more amino acid residues of said recombinant casein corresponding to one or more of the native phosphorylation sites.

4. The casein according to claim 1, wherein the recombinant casein has a degree of phosphorylation at each native phosphorylation site of at least 5%.

5. The system according to claim 1, wherein the microbial host cell is a bacterial cell, a fungal cell or a yeast cell.

6. The system according to claim 1, wherein the microbial host cell is a bacterial cell, which belongs to a genus selected from the group consisting of Lactococcus spp., Lactobacillus spp., Escherichia spp., Corynebacterium spp., Pseudomonas spp., Streptomyces spp. and Bacillus spp.; or
wherein the microbial host cell is a fungal host cell, which belongs to a genus selected from the group consisting of Kluyveromyces spp., Pichia spp., Saccharomyces spp., Tetrahymena spp., Yarrowia spp., Hansenula spp., Blastobotrys spp., Aspergillus spp., Candida spp., Zygosaccharomyces spp., Trichoderma spp., Chrysosporium spp., Fusarium spp., and Neurospora spp. and Debaryomyces spp.

7. The system according to claim 1, wherein the microbial kinase is a bacterial serine/threonine kinase or a yeast serine/threonine kinase and/or wherein the microbial kinase is a bacterial kinase of the Hanks family or a yeast kinase of the Hanks family.

8. The system according to claim 1, wherein the microbial kinase is selected from the group consisting of YabT (SEQ ID NO:5 or SEQ ID NO: 6), PrkD (SEQ ID NO: 7), PrkC (SEQ ID NO: 8) and PKNB1 (SEQ ID NO: 9) or functional variants thereof having at least 80% sequence identity, similarity or homology thereto.

9. The system according to claim 1, wherein the recombinant casein is selected from the group consisting of SEQ ID NO: 1, SEQ ID NO: 3, SEQ ID NO: 10, SEQ ID NO: 11, SEQ ID NO: 12, SEQ ID NO: 13, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a variant thereof having at least 90% sequence identity, similarity or homology thereto.

10. The system according to claim 1, wherein the recombinant casein is recombinant bovine casein or a recombinant human casein.

11. The system according to claim 10, wherein the recombinant bovine casein is selected from the group consisting of alpha-casein S1 (αS1), alpha-casein S2 (αS2), beta-casein and kappa-casein.

12. The system according to claim 1, wherein the recombinant casein is:
a. a recombinant bovine alpha-S1 casein (αS1) as set forth in SEQ ID NO: 1, is phosphorylated at least at one of the following positions: S46, S48, S64, S66, S67, S68, and S75;
b. a recombinant bovine alpha-S1 casein (αS1) as set forth in SEQ ID NO: 14, is phosphorylated at least at one of the following positions: S56, S61, S63, S79, S81, S82, S83, or S90;
c. a recombinant bovine alpha-S2 casein (αS2) as set forth in SEQ ID NO: 20, is phosphorylated at least at one of the following positions: S23, S24, S25, S28, S46, S71, S72, S73, S76, S144, S146, S150, or S158;
d. a recombinant bovine beta casein as set forth in SEQ ID NO: 3, is phosphorylated at least at one of the following positions: S15, S17, S18, S19 and S35;
e. a recombinant bovine beta casein as set forth in SEQ ID NO: 26, is phosphorylated at least at one of the following positions: S30, S32, S33, S34 and S50;
f. a recombinant bovine kappa casein as set forth in SEQ ID NO: 32, is phosphorylated at least at one of the following positions: S148, T166, S170, or S187.
g. a recombinant casein is selected from the group consisting of human alpha-casein S1 (αS1), human alpha-casein (αS2), human beta-casein and human kappa-casein.
h. a recombinant human alpha-S1 casein as set forth in SEQ ID NO: 10 is phosphorylated at least at one of the following positions: S31, S33, S41, S71, S85, S86, S88, S89, S90, or S91.
i. a recombinant human beta casein as set forth in SEQ ID NO: 22, is phosphorylated at least at one of the following positions: T18, S21, S23, S24, or S25.
j. a recombinant human kappa casein as set forth in SEQ ID NO: 28 is phosphorylated at positions T157.

13. The system according to claim 1, wherein said host cell comprises at least one protease deficiency rendering said host cell completely or partially deficient in the production and/or function of one or more proteases, and/or wherein said host cell expresses at least one chaperone protein.

14. A system for producing one or more phosphomimetic recombinant caseins, said system comprising a microbial host cell expressing:
i. at least one recombinant casein selected from the group consisting of alpha-casein S1 (αS1), alpha-casein S2 (αS2), beta-casein and kappa-casein, wherein one or more amino acid residues that are phosphorylated in a native mammalian casein are substituted with one or more phosphomimetic amino acids; and
ii. optionally at least one microbial kinase capable of phosphorylating said phosphomimetic casein at residues which are phosphorylated in a native mammalian casein, and which have not been substituted with phosphomimetic amino acids,
whereby said system is capable of producing one or more phosphomimetic recombinant casein.

15. The system according to claim 14, wherein one or more serine and/or threonine residues of the recombinant casein, such as of including alpha casein (αS1) as set forth in any one of SEQ ID NOs: 1, 10, 11, 12, 13 14 and 15, alpha casein (αS2) as set forth in any one of SEQ ID NOs: 16, 17, 18, 19, 20 and 21, beta casein as set forth in any one of SEQ ID NOs: 3, 22, 23, 24, 25, 26, and 27, and kappa casein as set forth in any one of SEQ ID NO 28, 29, 30, 31, 32 and 33, have been substituted with one or more phosphomimetic amino acids.

16. The system according to claim 14, wherein the recombinant casein is:
a. a recombinant casein is bovine alpha casein (αS1) as set forth in SEQ ID NO: 1, comprising a phosphomimetic amino acid at least at one of the following positions: S46, S48, S64, S66, S67, S68, and S75;
b. a recombinant casein is bovine alpha-S1 casein as set forth in SEQ ID NO: 14, comprising a phosphomimetic amino acid at least at one of the following positions: S56, S61, S63, S79, S81, S82, S83, or S90;
c. a recombinant casein is bovine alpha-S2 casein as set forth in SEQ ID NO: 20, comprising a phosphomimetic amino acid at least at one of the following positions: S23, S24, S25, S28, S46, S71, S72, S73, S76, S144, S146, S150, or S158;
d. a recombinant casein is bovine beta casein as set forth in SEQ ID NO: 3, comprising a phosphomimetic amino acid at least at one of the following positions: S15, S17, S18, S19 and S35;
e. a recombinant casein is bovine beta casein as set forth in SEQ ID NO: 26, comprising a phosphomimetic amino acid at least at one of the following positions: S30, S32, S33, S34 and S50;
f. a recombinant casein is bovine kappa casein as set forth in SEQ ID NO: 32, comprising a phosphomimetic amino acid at least at one of the following positions: S148, T166, S170, or S187;
g. a recombinant casein is human alpha-S1 casein as set forth in SEQ ID NO: 10, comprising a phosphomimetic amino acid at least at one of the following positions: S31, S33, S41, S71, S85, S86, S88, S89, S90, or S91;
h. a recombinant casein is human beta casein as set forth in SEQ ID NO: 22, comprising a phosphomimetic amino acid at least at one of the following positions: T18, S21, S23, S24, or S25;
i. a recombinant casein is human kappa casein as set forth in SEQ ID NO: 28, comprising a phosphomimetic amino acid at position T157.

17. The system according to claim 14, wherein each phosphomimetic amino acid is independently selected from the group consisting of aspartic acid, glutamic acid and/or combinations thereof.

18. The system according to claim 14, wherein the one or more phosphomimetic recombinant casein is as set forth in SEQ ID NO: 2 or SEQ ID NO. 4, or a variant thereof having at least 90% sequence identity, similarity or homology thereto.

19. A method of producing recombinant casein in a microbial host cell, comprising the steps of:
i. providing the system according to claim 1, wherein said host cell is capable of producing recombinant casein;
ii. incubating said host cell in a medium, under conditions allowing growth of the host cell, whereby at least one recombinant casein is produced; and
iii. incubating the at least one casein with at least one microbial kinase,
whereby phosphorylated recombinant casein is produced.

20. A method of producing recombinant casein in a microbial host cell, comprising the steps of:
i. providing the system according to claim 2, wherein said host cell is capable of producing recombinant casein;
ii. incubating said host cell in a medium, under conditions allowing growth of the host cell, whereby at least one recombinant phosphomimetic casein is produced.
